(19) United States
(12) Patent Application Publication
(10) Pub. No.: US 2004/0242511 A1
(43) Pub. Date: Dec. 2, 2004

(54) Method

(76) Inventors: Elizabeth Mary Claire Fisher, London (GB); Sarah Elizabeth Lloyd, London (GB); John Collinge, Queen Square (GB)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/470,014

(22) PCT Filed: Jan. 22, 2002
(86) PCT No.: PCT/GB02/00256

(30) Foreign Application Priority Data
Jan. 23, 2001 (GB) 0101763.1

(51) Int. Cl. A61K 49/00; A61K 48/00

(52) U.S. Cl. 514/44; 424/9.2

(57) ABSTRACT

The invention relates to a method for the detection of prions in a sample comprising the steps of contacting one or more test animals with the sample; incubating the test animals; monitoring the test animals for adverse effects or death; and optionally performing a biopsy on the test animals that display adverse effects or death for evidence of prions; wherein the test animals have prion incubation times of 196 days or less.

### FIELD OF INVENTION

The present invention relates to a method. In particular, the present invention relates to a method for the detection of prions in a sample.

### BACKGROUND TO THE INVENTION

By way of background information, a prion protein (PrP) is a transmissable particle devoid of nucleic acid. The PrP gene encodes prion proteins. The most notable prion diseases are Bovine Spongiform Encephalopathy (BSE), Scrapie of Sheep and Creutzfeldt-Jakob Disease (CJD) of humans. The most common manifestation of CJD is sporadic CJD (sCJD) which occurs spontaneously in individuals. Iatrogenic CJD (iCJD) is a disease that results from accidental infection. Familial CJD (fCJD) is a form of CJD that occurs rarely in families and is caused by mutations of the human PrP gene. Gerstmann-Strassler-Scheinker Disease (GSS) is an inherited form of human prion disease and the disease occurs from an autosomal dominant disorder. ‘New variant’ CJD (vCJD) of humans is a distinct strain type of CJD that is associated with a pattern of PrP glycoforms that are different from those found for other types of CJD. It has been suggested that BSE may have passed from cattle resulting in vCJD in humans.

Mice are often used to study the transmissibility of prion infection. Prions are injected into mice, which are then monitored to establish if the mice show clinical symptoms of prion infection. Transmission of mouse adapted prion strains to mice is highly efficient, displaying short incubation times e.g. 48±2 days (U.S. Pat. No. 6,008,435). Furthermore, almost 100% of the mice infected develop the disease and there is a very small standard error. For example, Stephenson et al. (2000) Genomics 69, 47-53 have studied the prion incubation times of the RML mouse prion isolate in two different strains of mice, CAST/Ei and SJL/J. The incubation times were 172±6 days and 105±4 days, respectively. Crosses between CAST/Ei and SJL/J mice resulted in mean prion incubation times of 129 days (range=58 to 173 days).

However, when transmitting prions from one species to another the situation is very different. A characteristic of prion diseases is that passage of prions between species is subject to prolonged incubation times. This is referred to as the “species barrier” and is problematic when attempting to detect prions from genetically distinct species using mice. For example, Scott et al. (1999), Proc. Natl. Acad.Sci. USA 96, 15137-15142 studied the susceptibility of transgenic mice to BSE and vCJD. The shortest incubation times were 217±6 days and 247±4 days.

WO 9704814 and U.S. Pat. No. 6,008,435 describe a method for detecting prions. Transgenic animals are used which are susceptible to prion infection from genetically diverse animals. This is achieved by using a PrP gene derived from a mouse with components of the PrP gene from the genetically diverse animal. The transgenic animal therefore has an artificial PrP gene and is susceptible to infection by human prions that cause sCJD, iCJD, fCJD or GSS. The shortest incubation time is 157±3 days in transgenic mice inoculated with prions that cause sCJD.

WO 9950404 describes the use of transgenic animals containing an exogenous PrP gene with an inducer sequence to effect expression of the PrP gene. These transgenic animals are susceptible to infection by human prions that cause sCJD, iCJD, fCJD or GSS such that symptoms of prion disease occur within 200 days.

Diagnostic assays to detect prions in a sample from bovines and humans that cause BSE and vCJD are limited by the species barrier. Consequently, when said samples are contacted with test animals, a test result cannot be obtained for 217±6 days to 247±4 days or even longer using the prior art methods. This is problematic since assay results need to supplied as rapidly as possible.

The present invention seeks to overcome problem(s) associated with the prior art.

### SUMMARY OF THE INVENTION

The present invention provides methods for the detection of prions in a sample. The methods are useful in detecting prions in said sample that cause BSE or vCJD in bovines and humans, respectively. The methods use a test animal, such as a SJL mouse in which clinical symptoms of prion infection are suprisingly displayed much more rapidly than using prior art methods. Typical incubation times for prion infection in the methods of the present invention are 196 days or less. This is a suprising finding because the species barrier between mice and bovines or mice and humans in prior art methods means that the time taken to display clinical symptoms is relatively long in such methods. Because the incubation time in the methods of the present invention is shorter than previously known, diagnosis of prion infection is significantly faster than prior art methods.

According to a first aspect, the present invention relates to a method for the detection of prions in a sample comprising the steps of: contacting one or more test animals with the sample; incubating the test animals; monitoring the test animals for adverse effects or death; and optionally performing a biopsy on the test animals that display adverse effects or death for evidence of prions; wherein the test animals have prion incubation times of 196 days or less.

Preferably, the test animals to be contacted with the sample are mice. More preferably, the test animals to be contacted with the sample are or are derived from, SJL mice. Most preferably, the test animals to be contacted with the sample are SJL mice.

The method according to the first aspect of the invention may further comprise the use of test animals which may be transgenic for one or more gene(s) from SJL mice. Preferably, the transgene(s) may comprise one or more PrP gene(s). More preferably, the PrP transgene(s) may encode a mammalian PrP. Most preferably, the PrP transgene(s) may encode a livestock or a human PrP.

Suitable test animals may include test animals that are transgenic for one or more prion susceptibility genes.

The methods of the present invention advantageously detect prions that cause BSE or vCJD in their appropriate host. Preferably, the sample that may contain prions is derived from a mammal. More preferably, the sample that may contain prions is derived from livestock or a human.

In a second aspect, the present invention relates to a method for the identification of genes associated with shorter prion incubation times comprising the steps of: contacting one or more test animals with a sample; incubating the test animals; monitoring the test animals for adverse effects or death; and optionally performing a biopsy on the test animals that display adverse effects or death for evidence of prions; identifying test animals with short and with longer prion incubation times; comparing the genes of test animals with short and with longer prion incubation times; identifying one or more genes that differ between test animals with short and with longer prion incubation times; and optionally determining the function of one or more genes that differ between test animals with short and with longer prion incubation times.

Comparison of genes of susceptible and non-susceptible animals may include comparison of gene structure (e.g. presence or absence or sequence determination or physical characteristics such as SNPs or other suitable techniques) and/or comparison of gene expression (e.g. using differential display, subtractive hybridisation or other suitable techniques).

In a third aspect, the present invention relates to a method for identifying one or more agent(s) capable of modulating prion infection comprising the steps of: contacting one or more test animals with a sample; contacting one or more test animals with an agent; incubating the test animals; monitoring the test animals for adverse effects or death; and optionally performing a biopsy on the test animals that display adverse effects or death for evidence of prions; identifying said agents that increase or decrease the prion incubation time.

Thus in another aspect, the present invention relates to an agent capable of modulating prion infection. Said agent may be advantageously used in the preparation of a pharmaceutical composition. Thus, in another aspect, the invention relates to modulation of prion infection in a subject by administering to said subject a therapeutically effective amount of said agent.

In a fourth aspect, the present invention relates to a method for estimating the amount of prions in a sample comprising the steps of: contacting one or more test animals with the sample; incubating the test animals; monitoring the test animals; noting the amount of time taken for the test animal to display clinical symptoms of prion infection and the amount of time taken for the test animal to die; estimating the amount of prions in the sample from said times.

In a fifth aspect, the present invention relates to a method of estimating the susceptibility of test animals to prion infection, comprising the steps of: contacting one or more test animals with a sample containing prions; incubating the test animals; monitoring the test animals for adverse effects or death; performing a biopsy on the test animals that display adverse effects or death for evidence of prions; performing glycoform ratio analysis; estimating the susceptibility of test animals to prion infection.

Preferably, when the sample contains prions, said prions are prions that cause BSE or vCJD in their appropriate host.

The test animals used in the present invention preferably have prion incubation times of 196 days or less. More preferably, the test animals have prion incubation times of 100 days or less. Most preferably, the test animals have prion incubation times of 40 days or less.

Advantages

The present invention has a number of advantages. These advantages will be apparent in the following description.

By way of example, the present invention is advantageous since it provides a commercially useful method.

By way of further example, the present invention is advantageous since it provides a method to identify prions in a sample more rapidly than existing methods.

By way of further example, the present invention advantageously provides a method for the identification of genes associated with the susceptibility of test animals, mammals, livestock or humans to prions.

By way of further example, the present invention advantageously provides for the identification of one or more agents capable of modulating prion infection and for the preparation of pharmaceutical composition(s) for the treatment of prion infection comprising said agents.

### DETAILED DESCRIPTION OF THE INVENTION

Prion

As used herein the term “prion” refers to a proteinaceous infectious particle that lacks nucleic acid.

The term “prion” is a term synonymous with the term “prion protein (PrP)”.

In a preferred embodiment of the present invention, samples are tested that may contain prions. When said samples contain prions, said prions are preferably prions that cause BSE or vCJD in their appropriate host.

Background teachings on prions have been presented by Victor A. McKusick et al on http://www.ncbi.nlm.nih.gov/Omim. The following information concerning prions has been extracted from that source:

Mutations in the prion protein gene are associated with Gerstmann-Straussler disease (GSD), Creutzfeldt-Jakob disease (CJD), and familial fatal insomnia, and aberrant isoforms of the prion protein can act as an infectious agent in these disorders as well as in kuru and in scrapie in sheep.

Prusiner (1982, 1987) suggested that prions represent a new class of infectious agent that lacks nucleic acid. (The term prion, which was devised by Prusiner (1982), comes from ‘protein infectious agent’) The prion diseases are neurodegenerative conditions transmissible by inoculation or inherited as autosomal dominant disorders. Prusiner (1994) reviewed the pathogenesis of transmissible spongiform encephalopathies and noted that a protease-resistant isoform of the prion protein was important in the pathogenesis of these diseases. Mestel (1996) reviewed the evidence for and against—and the opinions for and against—the existence of infectious proteins.

Tagliavini et al. (1991) purified and characterized proteins extracted from amyloid plaque cores isolated from 2 patients of the Indiana kindred. They found that the major component of GSD amyloid was an 11-kD degradation product of PrP, whose N-terminus corresponded to the glycine residue at position 58 of the amino acid sequence deduced from the human PrP cDNA. In addition, amyloid fractions contained larger PrP fragments with apparently intact N termini and amyloid P components. Tagliavini et al. (1991) interpreted these findings as indicating that the disease process leads to proteolytic cleavage of PrP, generating an amyloidogenic peptide that polymerizes into insoluble fibrils. Since no mutations of the structural gene were found in the family, factors other than the primary structure of PrP may play a crucial role in the process of amyloid formation.

One interpretation has been that the prion is a sialoglycoprotein whose synthesis is stimulated by the infectious agent that is the primary cause of this disorder and Manuelidis et al. (1987) presented evidence suggesting that the PrP peptide is not the infectious agent in CJD. Pablos-Mendez et al. (1993) reviewed the ‘tortuous history of prion diseases’ and suggested an alternative to the idea that prions are infectious, namely, that they are cytotoxic metabolites. The authors suggested that studies of the processing of the metabolite PrP and trials of agents that enhance the appearance of this protein would be useful ways to test their hypothesis. Their model predicted that substances capable of blocking the catabolism of PrP would lead to its accumulation. Increasing PrP synthesis in transgenic mice shortens the latency in experimental scrapie. The hypothesis of Pablos-Mendez et al. (1993) suggested an intracellular derailment of the degradative rather than the synthetic pathway of PrP.

Forloni et al. (1993) found that the PrP peptide 106-126 has a high intrinsic ability to polymerize into amyloid-like fibrils in vitro. They also showed that neuronal death results from chronic exposure of primary rat hippocampal cultures to micromolar concentrations of a peptide corresponding to this peptide. They suggested that the neurotoxic effect of the peptide involves an apoptotic mechanism.

It has been suggested that the infectious, pathogenic agent of the transmissible spongiform encephalopathies is a protease-resistant, insoluble form of the PrP protein that is derived posttranslationally from the normal, protease-sensitive PrP protein (Beyreuther and Masters, 1994). Kocisko et al. (1994) reported the conversion of normal PrP protein to the protease-resistant PrP protein in a cell-free system composed of purified constituents. This selective conversion from the normal to the pathogenic form of PrP required the presence of preexisting pathogenic PrP. The authors showed that the conversion did not require biosynthesis of new PrP protein, its amino-linked glycosylation, or the presence of its normal glycosylphosphatidylinositol anchor. This provided direct evidence that the pathogenic PrP protein can be formed from specific protein-protein interactions between it and the normal PrP protein.

Rivera et al. (1989) described a 13-year-old male with a severe progressive neurologic disorder whose karyotype showed a pseudodicentric chromosome resulting from a telomeric fusion 15p;20p. In lymphocytes the centromeric constriction of the abnormal chromosome was always that of chromosome 20, whereas in fibroblasts both centromeres were alternately constricted. The authors suggested that centromere inactivation results from a modified conformation of the functional DNA sequences preventing normal binding to centromere-specific proteins. They also postulated that the patient's disorder, reminiscent of a spongy glioneuronal dystrophy as seen in Creutzfeldt-Jakob disease, may be secondary to the presence of a mutation in the prion protein.

Collinge et al. (1990) suggested that ‘prion disease’, whether familial or sporadic, may prove to be a more appropriate diagnostic term. An Indiana kindred with GSD disease was reported by Farlow et al. (1989) and Ghetti et al. (1989). Using PrP gene analysis in genetic prediction carries potential problems arising out of uncertainty about penetrance and the complications of presymptomatic testing in any inherited late-onset neurodegenerative disorder. Collinge et al. (1991) concluded, however, that it had a role to play in improving genetic counseling for families with inherited prion diseases, allowing presymptomatic diagnosis or exclusion of CJD or GSD in persons at risk.

Gajdusek (1991) provided a chart of the PRNP mutations found to date: 5 different mutations causing single amino acid changes and 5 insertions of 5, 6, 7, 8, or 9 octapeptide repeats. He also provided a table of 18 different amino acid substitutions that have been identified in the transthyretin gene (TTR; 176300) resulting in amyloidosis and drew a parallel between the behavior of the 2 classes of disorders.

Schellenberg et al. (1991) sought the missense mutations at codons 102, 117, and 200 of the PRNP gene, as well as the PRNP insertion mutations, which are associated with CJD and GSSD, in 76 families with Alzheimer disease, 127 presumably sporadic cases of Alzheimer disease, 16 cases of Down syndrome, and 256 normal controls; none was positive for any of these mutations. Jendroska et al. (1994) used histoblot immunostaining in an attempt to detect pathologic prion protein in 90 cases of various movement disorders including idiopathic Parkinson disease (PD; 168600), multiple system atrophy, diffuse Lewy body disease (127750), Steele-Richardson-Olszewski syndrome (260540), corticobasal degeneration, and Pick disease (172700). No pathologic prion protein was identified in any of these brain specimens, although it was readily detected in 4 controls with Creutzfeldt-Jakob disease. Perry et al. (1995) used SSCP to screen for mutations at the prion locus in 82 Alzheimer disease patients from 54 families (including 30 familial cases), as well as in 39 age-matched controls. They found a 24-bp deletion around codon 68 which removed 1 of the 5 gly-pro rich octarepeats in 2 affected sibs and 1 offspring in a late-onset Alzheimer disease family. However, the other affected individuals within the same pedigree did not share this deletion, which was also detected in 3 age-matched controls in 6 unaffected members from a late-onset Alzheimer disease family. Another octarepeat deletion was detected in 3 other individuals from the same Alzheimer disease family, of whom 2 were affected. No other mutations were found. Perry et al. (1995) concluded that there was no evidence for association between prion protein mutations and Alzheimer disease in their survey.

Hsiao et al. (1990) found no mutation in the open reading frame of the PrP gene in 3 members of the family analyzed, but Hsiao et al. (1992) later demonstrated a phe198-to-ser mutation; see 176640.0011.

Palmer and Collinge (1993) reviewed mutations and polymorphisms in the prion protein gene.

Chapman et al. (1996) demonstrated fatal insomnia and significant thalamic pathology in a patient heterozygous for the pathogenic lysine mutation at codon 200 (176640.0006) and homozygous for methionine at codon 129 of the prion protein gene. They stressed the similarity of this phenotype to that associated with mutations in codon 178 (176640.0010).

Collinge et al. (1996) investigated a wide range of cases of human prion disease to identify patterns of protease-resistant PrP that might indicate different naturally occurring prion strain types. They studied protease resistant PrP from ‘new variant’ CJD to determine whether it represents a distinct strain type that can be differentiated by molecular criteria from other forms of CJD. Collinge et al. (1996) demonstrated that sporadic CJD and iatrogenic CJD (usually due to administration of growth hormone from cadaver brain) is associated with 3 distinct patterns of protease-resistant PrP on Western blots. Types 1 and 2 are seen in sporadic CJD and in some cases of iatrogenic CJD. A third type is seen in acquired prion diseases with a peripheral route of exposure to prions. Collinge et al.(l996) reported that ‘new variant’ CJD is associated with a unique and highly consisten appearance of protease-resistant PrP on Western blots involving a characteristic pattern of glycosylation of the PrP. Transmission of CJD to inbred mice produced a PrP pattern characteristic of the inoculated CJD. Transmission of bovine spongiform encephalopathy (BSE) prion produced a glycoform ratio pattern of PrP closely similar to that of ‘new variant’ CD. They found that the PrP from experimental BSE in macaques and naturally acquired BSE in domestic cats showed a glycoform pattern indistinguishable from that of experimental murine BSE and ‘new variant’ CJD. The report of Collinge et al. (1996) was reviewed by Aguzzi and Weissmann (1996), who concluded that Collinge et al. (1996) had reviewed the neuropathologic and clinical features of the 1 new variant’ of CJD that was related to BSE.

Prusiner (1996) provided a comprehensive review of the molecular biology and genetics of prion diseases. Collinge (1997) likewise reviewed this topic. He recognized 3 categories of human prion diseases: (1) the acquired forms include kuru and iatrogenic CJD; (2) sporadic forms include CJD in typical and atypical forms; (3) inherited forms include familial CJD, Gerstmann-Straussler-Scheinker disease, fatal familial insomnia, and the various atypical dementias. Collinge (1997) tabulated 12 pathogenetic mutations that had been reported to that time. Noting that the ability of a protein to encode a disease phenotype represents a nonmendelian form of transmission important in biology, Collinge (1997) commented that it would be surprising if evolution had not used this method for other proteins in a range of species. He referred to the identification of prion-like mechanisms in yeast (Wickner, 1994; Ter Avanesyan et al., 1994).

Horwich and Weissman (1997) reviewed the central role of prion protein in the group of related transmissible neurodegenerative diseases. The data demonstrated that prion protein is required for the disease process, and that the conformational conversion of the prion protein from its normal soluble alpha-helical conformation to an insoluble beta-sheet state is intimately tied to the generation of disease and infectivity. They noted that much about the conversion process remains unclear.

Mallucci et al. (1999) described a large English family with autosomal dominant segregation of presenile dementia, ataxia, and other neuropsychiatric features. Diagnoses of demyelinating disease, Alzheimer disease, Creutzfeldt-Jakob disease, and Gerstmann-Straussler-Scheinker syndrome had been made in particular individuals at different times. Mallucci et al. (1999) also described an Irish family, likely to be part of the same kindred, in which diagnoses of multiple sclerosis, dementia, corticobasal degeneration, and ‘new variant’ CJD had been considered in affected individuals. Molecular studies identified the disorder as prion disease due to an ala117-to-val mutation in the PRNP gene. They emphasized the diversity of phenotypic expression seen in these kindreds and proposed that inherited prion disease should be excluded by PRNP analysis in any individual presenting with atypical presenile dementia or neuropsychiatric features and ataxia, including suspected cases of ‘new variant’ CJD. Hegde et al. (1999) demonstrated that transmissible and genetic prion diseases share a common pathway of neurodegeneration. Hegde et al. (1999) observed that the effectiveness of accumulated PrP^{Sc}, an abnormally folded isoform, in causing neurodegenerative disease depends upon the predilection of host-encoded PrP to be made in a transmembrane form, termed CtmPrP. Furthermore, the time course of PrP^{Sc} accumulation in transmissible prion disease is followed closely by increased generation of CtmPrP. Thus, the accumulation of PrP^{Sc} appears to modulate in trans the events involved in generating or metabolizing CtmPrP. Hegde et al. (1999) concluded that together these data suggested that the events of CtmPrP-mediated neurodegeneration may represent a common step in the pathogenesis of genetic and infectious prion diseases.

PrP^{c}, the cellular, nonpathogenic isoform of PrP, is a ubiquitous glycoprotein expressed strongly in neurons. Mouillet-Richard et al. (2000) used the murine 1C11 neuronal differentiation model to search for PrP^{c}-dependent signal transduction thourough antibody-mediated crosslinking. The 1C11 clone is a committed neuroectodermal progenitor with an epithelial morphology that lacks neuron-associated functions. Upon induction, 1C11 cells develop a neural-like morphology, and may differentiate either into serotonergic or noradrenergic cells. The choice between the 2 differentiation pathways depends on the set of inducers used. Ligation of PrP^{c} with specific antibodies induced a marked decrease in the phosphorylation level of the tyrosine kinase FYN (137025) in both serotonergic and noradrenergic cells. The coupling of PrP^{c} to FYN was dependent upon caveolin-1 (601047). Mouillet-Richard et al. (2000) suggested that clathourin (see 118960) might also contribute to this coupling. The ability of the 1C11 cell line to trigger PrP^{c}-dependent FYN activation was restricted to its fully differentiated serotonergic or noradrenergic progenies. Moreover, the signaling activity of PrP^{c} occurred mainly at neurites. Mouillet-Richard et al. (2000) suggested that PrP^{c} may be a signal transduction protein.

Mapping

The human gene for prion-related protein has been mapped to 20p12-pter by a combination of somatic cell hybridization and in situ hybridization (Sparkes et al., 1986) and by spot blotting of DNA from sorted chromosomes (Liao et al., 1986). Robakis et al. (1986) also assigned the PRNP locus to 20p by in situ hybridization.

By analysis of interstitial 20p deletions, Schnittger et al. (1992) demonstrated the following order of loci: pter—PRNP—SCG1 (118920)—BMP2A (112261)—PAX1 (167411)—cen. Puckett et al. (1991) identified 5-prime of the PRNP gene a RFLP that has a high degree of heterozygosity, which might serve as a useful marker for the pter-p12 region of chromosome 20.

Riek et al. (1998) used the refined NMR structure of the mouse prion protein to investigate the structural basis of inherited human transmissible spongiform encephalopathies. In the cellular form of mouse prion protein, no spatial clustering of mutation sites was observed that would indicate the existence of disease-specific subdomains. A hydrogen bond between residues 128 and 178 provided a structural basis for the observed highly specific influence of a polymorphism at position 129 in human PRNP on the disease phenotype that segregates with the asp178-to-asn (D178N; 176640.0007) mutation. Overall, the NMR structure implied that only some of the disease-related amino acid replacements lead to reduced stability of the cellular form of PRNP, indicating that subtle structural differences in the mutant proteins may affect intermolecular signaling in a variety of different ways.

Windl et al. (1999) searched for mutations and polymorphisms in the coding region of the PRNP gene in 578 patients with suspect prion diseases referred to the German Creutzfeldt-Jakob disease surveillance unit over a period of 4.5 years. They found 40 cases with a missense mutation previously reported as pathogenic. Among these, the D178N mutation was the most common. In all of these cases, D178N was coupled with methionine at codon 129, resulting in the typical fatal familial insomnia genotype. Two novel missense mutations and several silent polymorphisms were found. In their FIG. 1, Windl et al. (1999) diagrammed the known pathogenic mutations in the coding region of PRNP.

History

Aguzzi and Brandner (1999) reviewed ‘the genetics of prions’ but raised the question of whether this is a contradiction in terms since the prion, which they defined as an enigmatic agent that causes transmissible spongiform encephalopathies, is a paradigm of nongenetic pathology. The protein-only hypothesis, originally put forward by Griffith (1967), says that prion infectivity is identical to scrapie protein, an abnormal form of the cellular protein, now referred to as PRNP. Replication occurs by the scrapie prion recruiting cellular prion and converting it into further scrapie prion. The newly formed scrapie prion will join the conversion cycle and lead to a chain reaction of events that results in an ever-faster accumulation of scrapie prion. This hypothesis gained widespread recognition and acceptance after Prusiner (1982) purified the pathologic protein and Weissmann and his colleagues (Oesch et al., 1985; Basler et al., 1986) cloned the gene that encodes the scrapie protein as well as its normal cellular counterpart PRNP. Even more momentum was achieved when Weissmann's group (Bueler et al., 1993) showed that genetic ablation of Prnp protects mice from experimental scrapie on exposure to prions, as predicted by the protein-only hypothesis. Aguzzi and Brandner (1999) considered the finding of linkage between familial forms of prion diseases and mutations in the prion gene to be an important landmark (Hsiao et al., 1989).

Animal Model

The structural gene for prion (Prn-p) has been mapped to mouse chromosome 2. A second murine locus, Prn-i, which is closely linked to Prn-p, determines the length of the incubation period for scrapie in mice (Carlson et al., 1986). Yet another gene controlling scrapie incubation times, symbolized Pid-1, is located on mouse chromosome 17. Quantitative trait loci (QTLs) affecting prion incubation time have also been described on Ch 9, 11 (Stephenson et al 2000), Ch 2, 11, 12 (Lloyd et al 2001), and Ch 2, 8, 4, 15 (Manolatron et al 2001). Scott et al. (1989) demonstrated that transgenic mice harboring the prion protein gene from the Syrian hamster, when inoculated with hamster scrapie prions, exhibited scrapie infectivity, incubation times, and prion protein amyloid plaques characteristic of the hamster. Hsiao et al. (1994) found that 2 lines of transgenic mice expressing high levels of the mutant P101L prion protein developed a neurologic illness and central nervous system pathology indistinguishable from experimental murine scrapie. Amino acid 102 in human prion protein corresponds to amino acid 101 in mouse prion protein; hence, the P101L murine mutation was the equivalent of the pro102-to-leu mutation (176640.0002) which causes Gerstmann-Straussler disease in the human. Hsiao et al. (1994) reported serial transmission of neurodegeneration to mice who expressed the P101L transgene at low levels and Syrian hamsters injected with brain extracts from the transgenic mice expressing high levels of mutant P101L prion protein. Although the high-expressing transgenic mice accumulated only low levels of infectious prions in their brains, the serial transmission of disease to inoculated recipients argued that prion formation occurred de novo in the brains of these uninoculated animals and provided additional evidence that prions lack a foreign nucleic acid.

Studies on PrP knockout mice have been reported by Bueler et al. (1994), Manson et al. (1994), and Sakaguchi et al. (1996). Sakaguchi et al. (1996) reported that the PrP knockout mice produced by them were apparently normal until the age of 70 weeks, at which point they consistently began to show signs of cerebellar ataxia Histologic studies revealed extensive loss of Purkinje cells in the majority of cerebellar folia. Atrophy of the cerebellum and dilatation of the fourth ventricle were noted. Similar pathologic changes were not noted in the PrP knockout mice produced by Bueler et al. (1994) and by Manson et al. (1994). Sakaguchi et al. (1996) noted that the difference in outcome may be due to strain differences or to differences in the extent of the knockout within the PrP gene. Notably, in all 3 lines of PrP knockout mice described, susceptibility to prion infection was lost.

Based on their studies in PrP null mice, Collinge et al. (1994) concluded that prion protein is necessary for normal synaptic function. They postulated that inherited prion disease may result from a dominant negative effect with generation of PrP^{Sc}, the posttranslationally modified form of cellular PrP, ultimately leading to progressive loss of functional PrP (PrP^{c}). Tobler et al. (1996) reported changes in circadian rhythm and sleep in PrP null mice and stressed that these alterations show intriguing similarities with the sleep alterations in fatal familial insomnia.

Mice devoid of PrP develop normally but are resistant to scrapie; introduction of a PrP transgene restores susceptibility to the disease. To identify the regions of PrP necessary for this activity, Shmerling et al. (1998) prepared PrP knockout mice expressing PrPs with amino-proximal deletions. Surprisingly, PrP with deletion of residues 32-121 or 32-134, but not with shorter deletions, caused severe ataxia and neuronal death limited to the granular layer of the cerebellum as early as 1 to 3 months after birth The defect was completely abolished by introducing 1 copy of a wildtype PrP gene. Shmerling et al. (1998) speculated that these truncated PrPs may be nonfunctional and compete with some other molecule with a PrP-like function for a common ligand.

Telling et al. (1996) reported observations that supported the view that the fundamental event in prion diseases is a conformational change in cellular prion protein whereby it is converted into the pathologic isoform PrP^{Sc}. They found that in fatal familial insomnia (FFI), the protease-resistant fragment of PrP^{Sc} after deglycosylation has a size of 19 kD, whereas that from other inherited and sporadic prion diseases is 21 kD. Extracts from the brains of FFI patients transmitted disease to transgenic mice expressing a chimeric human-mouse PrP gene about 200 days after inoculation and induced formation of the 19-kD PrP^{Sc} fragment, whereas extracts from the brains of familial and sporadic Creutzfeldt-Jakob disease patients produced the 21-kD PrP^{Sc} fragment in these mice. The results of Telling et al. (1996) indicated that the conformation of PrP^{Sc} functions as a template in directing the formation of nascent PrP^{Sc} and suggested a mechanism to explain strains of prions where diversity is encrypted in the conformation of PrP^{Sc}.

Lindquist (1997) pointed out that ‘some of the most exciting concepts in science issue from the unexpected collision of seemingly unrelated phenomena.’ The case in point she discussed was the suggestion by Wickner (1994) that 2 baffling problems in yeast genetics could be explained by an hypothesis similar to the prion hypothesis. Two yeast mutations provided a convincing case that the inheritance of phenotype can sometimes be based upon the inheritance of different protein conformations rather than upon the inheritance of different nucleic acids. Thus, yeast may provide important new tools for the study of prion-like processes. Furthermore, she suggested that prions need not be pathogenic. Indeed, she suggested that self-promoted structural changes in macromolecules lie at the heart of a wide variety of normal biologic processes, not only epigenetic phenomena, such as those associated with altered chromatin structures, but also some normal, developmentally regulated events.

Hegde et al. (1998) studied the role of different topologic forms of PrP in transgenic mice expressing PrP mutations that alter the relative ratios of the topologic forms. One form is fully translocated into the ER lumen and is termed PrP-Sec. Two other forms span the ER membrane with orientation of either the carboxy-terminal to the lumen (PrP-Ctm) or the amino-terminal to the lumen (PrP-Ntm). F2-generation mice harboring mutations that resulted in high levels of PrP-Ctm showed onset of neurodegeneration at 58±11 days. Overexpression of PrP was not the cause. Neuropathology showed changes similar to those found in scrapie, but without the presence of PrP^{Sc}. The level of expression of PrP-Ctm correlated with severity of disease.

Supattapone et al. (1999) reported that expression of a redacted PrP of 106 amino acids with 2 large deletions in transgenic (Tg) mice deficient for wildtype PrP (Prnp−/−) supported prion propagation. Rocky Mountain laboratory (RML) prions containing full-length PrP^{Sc} produced disease in Tg(PrP106)Prnp−/− mice after approximately 300 days, while transmission of RML106 prions containing PrP^{Sc106} created disease in Tg(PrP106)Prnp−/− mice after approximately 66 days on repeated passage. This artificial transmission barrier for the passage of RML prions was diminished by the coexpression of wildtype mouse PrP^{c} in Tg(PrP106)Prnp±mice that developed scrapie in approximately 165 days, suggesting that wildtype mouse PrP acts in trans to accelerate replication of RML106 prions. Purified PrP^{Sc106} was protease resistant, formed filaments, and was insoluble in nondenaturing detergents.

Kuwahara et al. (1999) established hippocampal cell lines from Prnp−/− and Prnp+/+ mice. The cultures were established from 14-day-old mouse embryos. All 6 cell lines studied belonged to the neuronal precursor cell lineage, although they varied in their developmental stages. Kuwahara et al. (1999) found that serum removal from the cell culture caused apoptosis in the Prnp−/− cells but not in Prnp+/+ cells. Transduction of the prion protein or the BCL2 gene suppressed apoptosis in Prnp−/− cells under serum-free conditions. Prnp−/− cells extended shorter neurites than Prnp+/+ cells, but expression of PrP increased their length. Kuwahara et al. (1999) concluded that these findings supported the idea that the loss of function of wildtype prion protein may partly underlie the pathogenesis of prion diseases. The authors were prompted to try transduction of the BCL2 gene because BCL2 had previously been shown to interact with prion protein in a yeast 2-hybrid system. Their results suggested some interaction between BCL2 and PrP in mammalian cells as well.

In scrapie-infected mice, prions are found associated with splenic but not circulating B and T lymphocytes and in the stroma, which contains follicular dendritic cells. Formation and maintenance of mature follicular dendritic cells require the presence of B cells expressing membrane-bound lymphotoxin-alpha/beta. Treatment of mice with soluble lymphotoxin-beta receptor results in the disappearance of mature follicular dendritic cells from the spleen. Montrasio et al. (2000) demonstrated that this treatment abolished splenic prion accumulation and retards neuroinvasion after intraperitoneal scrapie inoculation. Montrasio et al. (2000) concluded that their data provided evidence that follicular dendritic cells are the principal sites for prion replication in the spleen.

Chiesa et al. (1998) generated lines of transgenic mice that expressed a mutant prion protein containing 14 octapeptide repeats, the human homolog of which is associated with an inherited prion dementia. This insertion was the largest identified to that time in the PRNP gene and was associated with a prion disease characterized by progressive dementia and ataxia, and by the presence of PrP-containing amyloid plaques in the cerebellum and basal ganglia (Owen et al., 1992; Duchen et al., 1993; Krasemann et al., 1995). Mice expressing the mutant protein developed a neurologic illness with prominent ataxia at 65 or 240 days of age, depending on whether the transgene array was, respectively, homozygous or hemizygous. Starting from birth, mutant PrP was converted into a protease-resistant and detergent-insoluble form that resembled the scrapie isoform of PrP, and this form accumulated dramatically in many brain regions thouroughout the lifetime of the mice. As PrP accumulated, there was massive apoptosis of granule cells in the cerebellum.

Test Animal

As used herein, the term “test animal” refers to any animal useful in the methods of the present invention. The test animal may be any animal that is susceptible to infection by prions. Preferably, the test animal is a mammal. More preferably, the test animal is an adult mammal. More preferably, the test animal is a rat, hamster, rabbit, guinea pig or mouse. More preferably the test animal is a mouse. Most preferably the test animal is a SJL mouse.

As used herein, the term “SJL mouse” refers to a mouse of the SJL strain, or a mouse derived from the SJL strain. The term “mice derived from the SJL strain” is explained more fully below.

General biological information on SJL mice has been reviewed by Crispens (1973). SJL have a short life span when kept in conventional conditions (typically 472 days in males and 395 days in females). They have a high gross tumour incidence (Storer, 1966) with reticulum cell sarcomas (resembling Hodgkin's disease) occurring in about 90% of animals at an average age of about 13 months (Murphy, 1963; Crispens, 1973; Fujinaga et al., 1970, 1970). The unusual feature of the SJL reticulum cell tumours is their regular and early appearance, with the preneoplastic lesion detectable as early as 22 days (Potter, 1972). Tumour development as well as autoimmunity may result from an effective amplification of the immune response (Owens and Bonavida, 1976). SJL mice have a high incidence of spontaneous amyloidosis, possibly associated with fighting (Page and Glenner, 1972); develop gamma-1 and gamma-2 paraproteinaemia (Wanebo et al., 1966. 1966) and hyperplastic neuroretinopathy (Caffe et al. 1993).

In terms of physiology and biochemistry, SJL mice have low plasma cholesterol at 24 weeks (Weibust, 1973) and a high metabolic rate (Storer, 1967) with low serum ceruloplasmin levels in females and intermediate levels in males (Meier and MacPike, 1968). The strain also has high systolic blood pressure (Schlager and Weibust, 1967), low plasma cholinesterase activity in males (Angel et al., 1967), high mean heart rate (Blizard and Welty, 1971), high brain sphingosine and low brain sterol (Sampugna et al., 1975, 1975). Venous blood has a low pH (Dagg, 1966) and they are resistant to the development of atherosclerosis on a semi-synthetic high fat diet (Nishina et al, 1993). High intrinsic myogenicity of muscle cells both in vivo and in vitro (Maley et al, 1994, Mitchell et al, 1995) is also a feature.

The anatomy of SJL mice is such that it has a low brain (Storer, 1967), small spinal cord (Roderick et al., 1973, 1973) and the cerebellum has no intraculminate fissure between vermian lobule IV and vermian lobule V (the ventral and dorsal lobules of the culmen) (Cooper et al 1991). It has a low percentage of carcass lipid on a high-fat diet (West et al 1992) and a low retinal ganglion cell number (Williams et al, 1996). SJL mice also have a high bone density of the femur (Beamer et al, 1996).

SJL mice are resistant to induction of subcutaneous tumours by 3-methylcholanthrene (Kouri ET al., 1973, 1973), to X-irradiation (Roderick, 1963), to hyperbaric oxygen (Hill et al., 1968, 1968), to biliary tract injury following oral dosing with 500 micrograms of the fungal toxin sporidesmin (Bhathal et al 1990). They are susceptible to induction of splenic amyloidosis by injection of casein (Clerici, 1972), to induction of lymphoid and myeloid leukaemia by DMBA (Crispens, 1973), to ozone-induced decreases of tracheal potential (Takahashi et al, 1995), to weight loss induced by cocaine, but this is attenuated by anisomycin (Shimosato et al, 1994). Also, airways are hyporeactive to acetylcholine (Zhang et al, 1995) and it has a low voluntary consumption of morphine in a two-bottle choice situation (Belknap et al, 1993).

SJL mice are susceptible to induction of experimental allergic encephalomyelitis (EAE) (Levine and Sowinski, 1973) and has a low lymphocyte phytohaemagglutinin response (Heiniger et al., 1975, 1975). SJL mice have a poor immune response to small doses of bovine gamma-globulin (Levine and Vaz, 1970), to DNP-keyhole-limpet haemocyanin (Borel and Kilham, 1974), to (Pro66, Gly34) (Fuchs et al., 1974), no immune response to GAT (random terpolymer of Glu60, Ala30, Tyr10) (Dorf et al., 1974., 1974). The stain is very sensitive to anaphylactic shock (Treadwell, 1969) while they are resistant to induction of immunological tolerance (Fujiwara and Cinader, 1974). 1974). It has a high susceptibility to IgE- and IgG1-mediated passive cutaneous anaphylaxis (De Souza et al., 1974, 1974) and erythrocytes have a high agglutinability (Rubinstein et al., 1974., 1974). It has a low response to Dextran (Blomberg et al., 1972, 1972) and the immune response to type-III pneumococcal polysaccharide declines by 42 weeks, in contrast to BALB/c and C3H (Smith, 1976).

SJL mice are susceptible to induction of experimental autoimmune thyroiditis (Vladutiu and Rose, 1971a). Thymocytes exhibit a periodicity (5-9 days) in their response to hormonal stimulation with isoproterenol. This is expressed in large changes in the intensity of the response (peak levels of intracellular cAMP which vary approximately 6-fold), and in the response pattern, i.e., in the occurrence or non-occurrence of an immediate hormone-induced desensitization. They are resistant to immunosuppression of contact hypersensitivity by ultraviolet B light (Noonan and Hoffman, 1994) with low natural killer cell response to the immunostimulent 7-allyl-8-oxoguanosine (Pope et al, 1994). It has defective T cell receptor-induced interleukin-4 production and absence of T-cells with the NK1.1 antigen. However, natural-killer-like T-cells develop normally in spite of these defects (Beutner et al, 1997). Mast cells grow faster in culture and have high levels of histamine and TNF-alpha in their granules (Bebo et al, 1996). SJL mice also have a high level of serum complement C5 (Lynch and Kay, 1995).

Encephalomyocarditis virus causes diabetes mellitus in SJL mice (Boucher et al., 1975) and they have a high susceptibility to develop leukaemia on infection with Friend virus (Dietz and Rich, 1972). They are resistant to measles virus (Rager-Zisman et al., 1976) and develops flaccid paralysis whilst survivors develop a distinct neurological disorder associated with marked mononuclear cell infiltration and active demyelination in the spinal cord after intracerebral inoculation with Theiler's encephalomyelitis virus. The incubation period may be 2-3 months (Lipton and Dal Canto, 1976). They are resistant to street rabies virus (SRV) when injected via the intraperitoneal route (Perry and Lodmell 1991) and develops herpes simplex encephalitis (HSE) resembling the human condition, following intranasal infection with a neurovirulent clinical isolate of herpes simplex virus type 1 (Hudson et al 1991). They are resistant to carditis on infection with Lyme borreliosis (Borrelia burgdorferi) (Barthold et al 1990) and has high eosinophilia on infection with the helminth Mesocestoides corti and highly susceptible to infection with the parasite. Larval burdens at 21 days after infection with 100 tetrathyridia being considerably higher (greater than 1000) than all other strains except NIH, which was comparable (Lammas et al 1990). SJL mice are susceptible to infection by Helicobacter felis with moderate to severe chronic active gastritis in the body of the stomach, which increased over time (Sakagami et al, 1996).

Additional background teachings on SJL mice have been presented by Victor A. McKusick et al on http://www.ncbi.nlm.nih.gov/Omim. The following information concerning the SJL mice has been extracted from that source.

The SJL mouse strain (Festing, 1979) is susceptible to many induced autoimmune diseases such as experimental autoimmune encephalitis (EAE) and inflammatory muscle disease. Additionally, the skeletal muscle of SJL mice was shown to have an increased regenerative capacity and demonstrates the spontaneous occurrence of what was designated an ‘inflammatory myopathy,’ accompanied by loss of strength. By histopathologic examinations of muscles in SJL mice of different ages, Bittner et al. (1999) found features compatible with a progressive muscular dystrophy, including degenerative and regenerative changes of muscle fibres and a progressive fibrosis. Histologically, the changes were observed in mice as young as 3 weeks of age. Changes affected primarily the proximal muscle groups, whereas the distal muscles remained less affected. The morphologic alterations were associated with signs of slowly progressive muscle weakness, which Bittner et al. (1999) detected as early as 3 weeks after birth when mice were suspended by their tails. The phenotype was found to be inherited as an autosomal recessive trait and was found to map to mouse chromosome 6, in a region syntenic with human 2p13, where the DYSF gene maps. Because of this synteny, Bittner et al. (1999) studied dysferlin in these mice. They found a reduction to approximately 15% of control levels in SJL mice. They found a 171-bp deletion in the Dysf gene of SJL mice, predicted to result in removal of 57 amino acids, including most of the fourth C2 domain. The last C2 domain is conserved in other members of the fer-like gene family.

Mice Derived from the SJL Strain

As used herein, the term “mice derived from the SJL strain” refers generally to any mice that have a genetic background that may contain one or more genes of SJL mice.

Mice derived from SJL mice include any mice having an SJL ancestor. Preferably, mice derived from SJL mice have an SJL grandparent. More preferably, mice derived from SJL mice have an SJL parent.

Said mice may be derived from SJL mice by various methods of breeding including in-breeding, out-breeding or any other breeding method known to a person skilled in the art.

Mice derived from SJL mice may also include SJL mice carrying one or more transgenes. Said transgene(s) may be derived from SJL mice, from non-SJL mice, or from any other source.

In a preferred embodiment, the mice derived from SJL have an altered susceptibly to prion infection from genetically distinct species. Preferably, prion incubation times are decreased with respect to SJL mice.

Sample

The term “sample” as used herein, has its natural meaning. A sample may be any physical entity tested for the presence of prions according to the methods of the present invention. The sample may be or may be derived from biological material.

Said sample may be or may be derived from one of more entities selected from a swab, biopsy, brain homogenate, prion binding substance or object comprising same or any other suitable material.

Sample administration

The sample may be prepared by mixing with a solution. Preferably, the solution is a buffer such as phosphate buffered saline. The sample may be contacted with one or more test animals that have been anaethetised using an anaesthetic such as halothane/O₂. The sample may be contacted with the test animal via any suitable route such as via intraperitoneal (IP) or oral routes, or by direct introduction into nervous tissue such as the brain. When contacting mice with prions by intra-periotoneal inoculation, a preferred dose is 100 μl of 10% homogenate. When contacting mice with prions orally, a preferred dose is 10 g by feeding of, for example, BSE infected cow brain. Preferably, the method of contact is via introduction of at least part of the sample into the brain of the test animal, such as by injection. More preferably, the sample is injected into the right parietal lobe of the brain of said test animal. The test animal may be incubated following contact with the sample. As used herein, the term “incubated” means the maintenance of the test animal in appropriate conditions, such as a containment facility as is well known in the art.

Monitoring of Test Animal

Test animals may be monitored for symptoms of prion infection by examination for the development of symptoms of prion infection. At the onset of symptoms, the test animals are examined regularly and may be culled if showing signs of distress. Criteria for clinical diagnosis of prion infection in mice, including examples of symptoms are described by Carlson (1986), Cell, 46, 503-511 and include, among others, generalised tremor, ataxia or rigidity of the tail, or combinations thereof. Optionally, biopsies of the test animals may be performed. The biopsy may be performed on any suitable organ or tissue such as one in which prions accumulate. Preferably, a brain biopsy is performed. Various methods well known in the art may be used for the detection of prion proteins such as Western blotting (Collinge et al. 1996, Nature 383, 685-690), immunoassay (described in WO 9837210) and electronic-property probing (described in WO 9831839).

Adverse Effects

As used herein, the term “adverse effects” refers to the clinical signs of neurological dysfunction caused by prion infection, as discussed above. When clinical signs appear, the test animals are examined daily. If the death of one or more test animals is obviously imminent, their brains are removed for histopathologic studies and confirmation of prion infection.

Prion Incubation Time

As used herein, the term “prion incubation time” refers to the amount of time the elapses from contacting a test animal with a sample, to the time when the test animal first displays adverse effects or death resulting from prion infection.

The methods of the present invention advantageously posses short incubation times. This allows results to be obtained more rapidly and more economically than prior art methods. Preferably, the prion incubation time is 196 days or less. Preferably, the prion incubation time is 189 days or less. Preferably, the prion incubation time is 169 days or less. Preferably, the prion incubation time is 151 days or less. Preferably, the prion incubation time is 149 days or less. Preferably, the prion incubation time is 139 days or less. Preferably, the prion incubation time is 117 days or less. Preferably, the prion incubation time is 100 days or less. Preferably, the prion incubation time is 89 days or less. Preferably, the prion incubation time is 78 days or less. Preferably, the prion incubation time is 65 days or less. Preferably, the prion incubation time is 51 days or less. Preferably, the prion incubation time is 40 days or less.

In a preferred embodiment, the prion incubation times refer to the times elapsed from contacting a test animal with a sample containing prions capable of causing vCJD, or prions capable of causing BSE, to the time when the test animal first displays adverse effects or death resulting from prion infection. Preferably, the times refer to primary passage of prion samples. Further explanation may be found below, such as in the Examples section.

Attack Rate

As used herein, the term “attack rate” refers to the percentage of test animals contacted with an identical sample that display symptoms of prion infection. For example, if ten test animals are contacted with the same sample and four of them display symptoms of prion infection, then the attack rate is 40%. Low attack rates are a feature of the “species barrier” which is discussed below.

A person skilled in the art will be aware that it is necessary to use a sufficient number of test animals per sample in the methods of the present invention, taking the attack rate into account. Preferably, the number of test animals used is between 5 and 50. More preferably, the number of test animals used is between 15 and 40. Most preferably, the number of test animals used is between 20 and 30. In a preferred embodiment of the present invention, the attack rate is greater than 50%.

Species Barrier

As used herein, the term “species barrier” refers to one or more factor(s) limiting the transfer of prions between species.

An effect of the species barrier is prolonged prion incubation times during passage in to a new host (Pattison, 1965) even when transmission is attempted by the most efficient route. In contrast, same-species transmission of prions is normally highly efficient. A number of factors influence the species barrier between a donor and recipient including differences in the PrP sequence and the strain of prion. The PrP sequence is determined by the donor who therefore dictates the ‘species’ of the prion. The strain of prion appears to be determined by the conformation of PrP^{Sc} (Collinge et al. 1996). For example, CJD prions propagated in humans expressing wild-type PrP transmit highly efficiently to transgenic mice expressing human PrP. vCJD prions propagated and transmitted in the same way have transmission properties completely distinct from CJD prions (Collinge et al. 1995; Hill et al. 1997). Another factor that may also contribute to the species barrier is the species specificity of a protein initially called protein X (Telling et al. 1995). This protein may bind to PrP^{C} and facilitate PrP^{Sc} formation. One such protein has been described in U.S. Pat. No. 5,962,669 called Prion Protein Modulator Factor (PPMF). PPMF is species specific such that PPMF from one species of mammal will only bind to PrP^{C} of the same or genetically similar species.

The biological effect of a species barrier is to increase mean incubation periods, increase the range of incubation periods and reduce the fraction of animals succumbing to the disease (Hill et al. 1960). The most widely studied species barrier is that which limits transmission between hamsters and mice. For example, the hamster scrapie strain Sc237 (Hecker et al. 1992) is regarded as nonpathogenic for mice and has been used for various studies in transgenic mice. Transgenic mice expressing hamster PrP are highly susceptible to Sc237 hamster prions with short incubation periods. In a recent reevaluation of this species barrier it has been found that conventional mice inoculated with Sc237 hamster prions showed no clinical signs but accumulated high prion titers in the brain (Hill et al. 1960).

It is an advantage of the present invention that the effects of the species barrier are reduced. For example, in accordance with the present invention, prions can be efficiently transmitted to test animals such that the prion incubation time is reduced to 196 days or less.

Genetically Distinct Species

As used herein, the term “genetically distinct species” refers to those species between which a species barrier exists with respect to prion infection. Consequently, long prion incubation times exist when prions are transferred between genetically distinct species. For example, a mouse is a genetically distinct species with respect to a human or bovine. In prior art methods, detecting prions in a sample from a genetically distinct species using test animals such as prior art mouse strain(s) results in prion incubation times of greater than 217±6 days.

It is an advantage of the present invention that prion incubation time between genetically distinct species is 196 days or less.

Measuring Prion Levels

In another aspect, the invention relates to the estimation of the amount of prions within the sample. This is achieved by studying the time taken for test animals contacted with the sample of infective prions to show clinical symptoms and the time taken for test animals contacted with the sample of infective prions to die. Briefly, the time at which the test animals are contacted with the sample (and dilutions thereof) is recorded. The test animals are then monitored for the development of clinical symptoms. Criteria for clinical diagnosis of prion infection in mice is discussed above and is further described by Carlson et al. (1986), Cell, 46, 503-511. At the onset of clinical symptoms the time is recorded. The test animals are monitored again, initially on a daily basis and then, as death approaches, more frequently. When death occurs, the time is again recorded. The intervals between the onset of clinical symptoms and death are calculated. This time interval is inversely proportional to the amount of prions in the sample. The logarithms of the time intervals minus a time factor are linear functions of the logarithms of the numbers of prions in the sample. The time factor is determined by maximising the linear relationship between time interval and dose in accordance with Pruisner et al. (1982), Annals. of Neurology 11 353-358.

Transgenic Animals

As used herein, the term “transgenic animals” refers to those animals that have a gene in their genetic complement that has been originally introduced using recombinant DNA technology. Recombinant DNA technology is well known to a person skilled in the art. In transgenic animals, the term “gene” is synonymous with the term “transgene”.

The test animals of the present invention may be transgenic test animals. Preferably, said test animals may be transgenic rats, hamsters, rabbits, guinea pigs or mice. More preferably, the test animals may be transgenic mice. Most preferably the test animals may be transgenic SJL mice.

Transgene(s) may be introduced onto SJL background by breeding from other transgenic mice having transgene(s) on a different genetic background.

Exogenous PrP Genes

As used herein, the term “exogenous PrP genes” refers generally to PrP genes from any species, which encode any form of PrP amino acid sequence or protein, in an arrangement or context different from their natural arrangement or context. Some commonly known PrP sequences have been described by Gabriel et al. (1992), Proc. Natl. Acad. Sci. USA 89, 9097-9101. Accordingly, the term “exogenous PrP gene” is also used to encompass the terms “artificial PrP gene” and “chimeric PrP gene”. As used herein, the term's “artificial PrP gene” and “chimeric PrP gene” refer to genes constructed by recombinant DNA technology, using methods well known to a person skilled in the art. When exogenous PrP genes are included in the genome of an animal then it will render that animal susceptible to infection from prions that would naturally only infect a genetically distinct species. Transgenic animals containing artificial PrP genes are described in U.S. Pat. No. 5,792,901, U.S. Pat. No. 5,908,969, U.S. Pat. No. 6,008,435 and WO 9704814.

In a preferred aspect, the test animals may be SJL mice that are transgenic for one or more exogenous PrP genes. Preferably, the exogenous PrP genes encode a mammalian PrP. Most preferably, the exogenous PrP gene(s) encode a livestock or a human PrP.

Livestock

The term “livestock”, as used herein refers to any farmed animal. Preferably, livestock are one or more of a pig, sheep, cow or bull. More preferably, livestock are a cow or bull.

Regulatory Sequences

In some applications of the present invention, a polynucleotide is operably linked to a regulatory sequence which is capable of directing the expression of the coding sequence, such as in vivo in the test animal. By way of example, the present invention may involve the use of regulatory sequences operably linked to one or more transgenes to modulate their expression. The expression of one or more exogenous PrP genes may be modulated such that the prion incubation time is increased or decreased. Advantageously, the expression of one or more exogenous PrP gene(s) from bovines or humans may be modulated in a transgenic animal such as a SJL mouse.

Transgenic animals with inducible expression of exogenous PrP genes and their methods of use are disclosed in WO 99/50404.

The term “operably linked” refers to a juxtaposition wherein the components described are in a relationship permitting them to function in their intended manner. A regulatory sequence “operably linked” to a coding sequence is ligated in such a way that expression of the coding sequence is achieved under condition compatible with the control sequences.

The term “regulatory sequences” includes promoters and enhancers and other expression regulation signals.

The term “promoter” is used in the normal sense of the art, e.g. an RNA polymerase binding site.

Enhanced expression of the polynucleotide encoding a polypeptide may also be achieved by the selection of heterologous regulatory regions, e.g. promoter, secretion leader and terminator regions, which serve to increase expression and, if desired, secretion levels of the protein of interest from the chosen expression host and/or to provide for the inducible control of the expression of a polypeptide.

The nucleotide sequence(s) may be operably linked to at least a promoter.

Aside from the promoter native to the gene encoding a polypeptide, other promoters may be used to direct expression of such a polypeptide. The promoter may be selected for its efficiency in directing the expression of such a polypeptide in the desired expression host.

In another embodiment, a constitutive promoter may be selected to direct the expression of a particular polypeptide. Such an expression construct may provide additional advantages since it circumvents the need to culture the expression hosts on a medium containing an inducing substrate.

Examples of strong constitutive and/or inducible promoters which are preferred for use in fungal expression hosts are those which are obtainable from the fungal genes for xylanase (xlnA), phytase, ATP-synthetase, subunit 9 (oliC), triose phosphate isomerase (tpi), alcohol dehydrogenase (AdhA), α-amylase (amy), amyloglucosidase (AG—from the glaA gene), acetamidase (amdS) and glyceraldehyde-3-phosphate dehydrogenase (gpd) promoters.

Examples of strong yeast promoters are those obtainable from the genes for alcohol dehydrogenase, lactase, 3-phosphoglycerate kinase and triosephosphate isomerase.

Examples of strong bacterial promoters are the α-amylase and SP02 promoters as well as promoters from extracellular protease genes.

Hybrid promoters may also be used to improve inducible regulation of the expression construct.

The promoter may additionally include features to ensure or to increase expression in a suitable host For example, the features may be conserved regions such as a Pribnow Box or a TATA box. The promoter may even contain other sequences to affect (such as to maintain, enhance, decrease) the levels of expression of the nucleotide sequence. For example, suitable other sequences include the Sh1-intron or an ADH intron. Other sequences include inducible elements—such as temperature, chemical, light or stress inducible elements. Also, suitable elements to enhance transcription or translation may be present. An example of the latter element is the TMV 5′ signal sequence (see Sleat (1987), Gene 217, 217-225; and Dawson (1993), Plant Mol. Biol. 23, 97).

Preparation of Transgenic Animals

Transgenes may be introduced in to test animals using several techniques well known to a person skilled in the art, such as: (1) the transfer of cells from one embryo to another; (2) introduction of cells infected with a retrovirus; (3) microinjection of cDNA into the pronucleus of a fertilised egg according to the methods described by Scott et al. (1989), Cell 59, 847-857 and Scott et al. (1992) Protein Sci., 1, 986-997; (4) implantation of multiple eggs into a single animal. Known procedures are then used to determine whether the resulting offspring are transgenic animals.

As used herein, the term “transgenic animal” also refers to knock-out mice which are well known to persons skilled in the art

In a preferred embodiment of the present invention the test animal may be transgenic for one or more genes. A transgenic animal with more than one transgene may be achieved by crossbreeding a first animal transgenic for one gene with a second animal transgenic for a second gene according to methods well known in the art. For example, the genetic background of an original transgenic mouse can be changed to SJL by breeding techniques such as by making a congenic. Congenic mice are typically produced by 10 generations of backcrossing. For example, a transgenic mouse could be made on a FvB background and then changed to an SJL background by this technique.

Clearly, it may be desirable to prepare transgenic animals comprising the prion susceptibility genes of SJL mice identified according to the present invention. In this manner, transgenic animals with increased or decreased prion susceptibility could be prepared.

Identification of Genes

In another embodiment, the present invention relates to methods for the identification of genes associated with the susceptibility of test animals to prions. As used herein, the term ‘identification of genes’ relates to the identification of the nucleic acids that comprise prion susceptibility genes.

The genes of susceptible (e.g. SJL) and non-susceptible (e.g. C57B16) mice may be compared by various methods known to persons skilled in the art, such as expression analysis by microarray, subtractive hybridisation (Sambrook et al. (1989) Konietzko U & Kuhl D (1998). Nucleic Acids Res 26, 1359-61) and genome scanning (Stephenson et al. (2000) Genomics 69, 47-53). By using these methods, regions of DNA that are different between non-susceptible and susceptible mice are identified. The genes involved in the susceptibility of certain strains of mice, such as SJL; to prions that cause BSE and vCJD in their appropriate hosts may then be identified.

A mapping method for identification of prion susceptibility genes would be to generate a cross between SJL and C57BL/6 eg F2 intercross or backcross. All animals from the cross would be inoculated with prions to determine their incubation time and genotyped by with microsatellite. Linkage analysis would identify candidate regions. Standard positional cloning techniques would then be used (eg. use of bioinformatics to identify candidate genes, sequencing, analysis of expression levels—see also below).

For a non-positional approach, it is possible to employ hybridisation to commercially available microarrays. For example, total RNA can be prepared from at least three SJL mice and three C57BL/6 mice (preferably from brain or spleen). RNA may be isolated using a commercially available kit such as RNAwiz™ from Ambion. cRNA is prepared and hybridized to arrays (eg Affymetrix mouse Genechips) in order to identify genetic differences between susceptible and non-susceptible mice.

In subtractive hybridisation, cDNA is prepared from susceptible and non-susceptible mice and processed so that the cDNA becomes highly enriched for sequences present only in the susceptible mice. The enriched cDNA may then be used to screen a cDNA library for clones that have sequences homologous to those of the enriched cDNA. The screening may be performed using a number of methods well known in the art such as nucleic acid hybridisation or with PCR probes.

The specificity of the probe i.e. whether it is derived from a highly conserved, conserved or non-conserved region and the stringency of the hybridisation or amplification (high, intermediate or low) will determine whether the probe identifies only naturally occurring coding sequences, or related sequences.

In genomic screening, genomic DNA from repeating DNA segments (called microsatellite DNA) is amplified by PCR from susceptible and non-susceptible mice. Linkage analysis is then performed to determine the relative positions of the genes on the chromosomes.

Preferably, the PCR primers used for amplifying microsatellite DNA are selected from commercially available kits such as the mouse genome-wide screening set (Research Genetics, Huntsville, Ala.). PCR reactions are performed in 96 well plates using for example, radioactively or fluorescently labeled primers. The PCR products are then resolved by denaturing gel electrophoresis and the products detected using for example, autoradiography or using an automated sequencer or capillary sequencer. Marker genes that have known locations on chromosomes are compared in susceptible and non-susceptible mice and those that show suggestive or significant linkage are subjected to linkage analysis. This can be employed using a suitable software package such as Map Manager QT (Manly & Olson et al. 1999, Mamm. Genome 10 327-334). The location of any genes that differ between susceptible and non-susceptible mice can then mapped.

PCR as described in U.S. Pat. No. 4,683,195, U.S. Pat. No. 4,800,195 and US 4965188 provides additional uses for oligonucleotides based upon target sequences. Such oligomers are generally chemically synthesized, but they may be generated enzymatically or produced from a recombinant source. Oligomers generally comprise two nucleotide sequences, one with sense orientation (5′->3′) and one with antisense (3′<-5′) employed under optimised conditions for identification of a specific gene or condition. The same two oligomers, nested sets of oligomers, or even a degenerate pool of oligomers may be employed under less stringent conditions for detection and/or quantification of closely related DNA or RNA sequences.

Probes may also be used for mapping the endogenous genomic sequence of the prion susceptibility genes, or for bioinformatic purposes. The sequences may be mapped to a particular chromosome or to a specific region of the chromosome using well known techniques. These include in situ hybridisation to chromosomal spreads (Verma et al (1988) Human Chromosomes: A Manual of Basic Techniques, Pergamon Press, New York City), flow-sorted chromosomal preparations, or artificial chromosome constructions such as YACs, bacterial artificial chromosomes (BACs), bacterial PI constructions or single chromosome cDNA libraries.

In situ hybridisation of chromosomal preparations and physical mapping techniques such as linkage analysis using established chromosomal markers are invaluable in extending genetic maps. Examples of genetic maps can be found in Science (1995; 270:410f and 1994; 265:1981f). Often the placement of a gene on the chromosome of another species may reveal associated markers even if the number or arm of a particular human chromosome is not known. New sequences can be assigned to chromosomal arms or parts thereof, by physical mapping. This provides valuable information when searching for prion susceptibility genes using positional cloning or other gene discovery techniques. Once the prion susceptibility genes have been crudely localised by genetic linkage to a particular genomic region any sequences mapping to that area may represent associated or regulatory genes for further investigation.

Optionally, the function of the prion susceptibility genes could be determined by first sequencing the DNA of the said gene(s) using methods well known in the art. DNA may be isolated from strains of mice by taking a section of tail and incubating it overnight at 55° C. in 0.7 ml of buffer (50 mM Tris-HCl pH 8.0, 100 mM EDTA, 100 mM NaCl, 1% SDS) containing 500 μg/ml proteinase K. The samples are then extracted with phenol:chloroform. High molecular weight DNA is spooled onto glass pipettes after ethanol precipitation and redissolved in TE buffer (10 mM Tris, 1 mM EDTA, pH 7.5). Advantageously, DNA isolation may be accomplished using a suitable commercially available DNA extraction kit such as those supplied by Promega (using nuclei lysis buffer followed by protein precipitation buffer), thereby avoiding the need for phenol-chloroform extraction. The sequence(s) could then be compared with DNA sequences available on databases to identify homologous sequences. Using this information, the function of the homologous sequences could be determined.

Modulating Prion Infection

The term “modulating” may refer to preventing, suppressing, alleviating, restorating or elevating or otherwise affecting a prion infection in a mammal.

Glycoform Ration Analysis

As used herein, the term “glycoform ratio analysis” refers to the analysis of the different types of glycosylation of PrP^{Sc} i.e. the di-, mono- and un-glycosylated forms, as described by Baron et al. (1999) J. Clin. Microbiol. 37, 3701-3704. The molecular weight and relative intensity of each of the three glycoforms may be measured to calculate a glycoform ratio. For example, BSE and vCJD passaged into FVB and Tg152 mice give a characteristic glycoform ratio where the di-glycosylated band is stained more intensely.

Estimating the Susceptibility of Test Animals to Prion Infection

Advantageously, the methods of the present invention may be used to estimate the susceptibility of test animals to prion infection. One or more test animals may be contacted with a sample containing prions. Preferably, the prions are prions which would cause BSE or vCJD in their appropriate host. The test animals may then be incubated and monitored for adverse effects or death. A biopsy on the test animals that display adverse effects or death may be performed for evidence of prions. Preferably, a brain biopsy is performed. Glycoform ratio analysis may then be performed according to Baron et al. (1999) J. Clin. Microbiol. 37, 3701-3704. Briefly, the brain is homogenised. The brain homogenate is incubated with a proteinase such as Proteinase K. Following centrifugation, pellets are resuspended in a denaturing buffer. The proteins are then be separated by SDS-PAGE. Western blotting is performed using antisera raised against PrP from an appropriate host that cross-reacts with all three glycoforms: Bound antibodies may then be detected using a variety of methods well known to a person skilled in the art. Preferably, three separate SDS-PAGE gels are Western blotted for each individual. The relative intensity of each of the three glycoforms may then be estimated. If the glycoform ratio analysis shows that the mono-glycosylated form is most dominant, then it may be estimated that the test animals have prion incubation times of 241±15 days or less. If the glycoform ratio analysis shows that the mono-glycosylated form is not the most dominant, then it may be estimated that the test animals have prion incubation times of 242±15 days or more. Optionally, the exact prion incubation time of test animals that have a dominant mono-glycosylated form may be determined using the methods described in the present invention.

Treatment

It is to be appreciated that all references herein to treatment refer to the modulation of prion infection.

The treatment may be of mammals such as livestock and/or humans.

Agent

As used herein, the term “agent” may be a single entity or it may be a combination of entities.

The agent may be an organic compound or other chemical. The agent may be a compound, which is obtainable from or produced by any suitable source, whether natural or artificial. The agent may be an amino acid molecule, a polypeptide, or a chemical derivative thereof, or a combination thereof. The agent may even be a polynucleotide molecule—which may be a sense or an anti-sense molecule. The agent may even be an antibody.

The agent may be designed or obtained from a library of compounds, which may comprise peptides, as well as other compounds, such as small organic molecules.

By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetics, a derivatised agent, a peptide cleaved from a whole protein, or a peptides synthesised synthetically (such as, by way of example, either using a peptide synthesizer or by recombinant techniques or combinations thereof, a recombinant agent, an antibody, a natural or a non-natural agent, a fusion protein or equivalent thereof and mutants, derivatives or combinations thereof.

Typically, the agent will be an organic compound. Typically the organic compounds will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the agent comprises at least one cyclic group. The cyclic group may be a polycyclic group, such as a non-fused polycyclic group. For some applications, the agent comprises at least the one of said cyclic groups linked to another hydrocarbyl group.

The agent may contain halo groups. Here, “halo” means fluoro, chloro, bromo or iodo.

The agent may contain one or more of alkyl, alkoxy, alkenyl, alkylene and alkenylene groups—which may be unbranched- or branched-chain.

The agent may be in the form of a pharmaceutically acceptable salt—such as an acid addition salt or a base salt—or a solvate thereof, including a hydrate thereof. For a review on suitable salts see Berge et al, J. Pharm. Sci., 1977, 66, 1-19.

The agent of the present invention may be capable of displaying other therapeutic properties.

The agent may be used in combination with one or more other pharmaceutically active agents.

If combinations of active agents are administered, then they may be administered simultaneously, separately or sequentially.

Amino Acid Sequence

Aspects of the present invention concern the use of amino acid sequences, which are available in databases. These amino acid sequences may comprise the agent of the present invention. In another embodiment, the amino acid sequences may be used as a target to identify suitable agents for use in the composition of the present invention. In another embodiment, the amino acid sequences may be used as a target to verify that an agent may be used as an agent according to the present invention.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

The amino acid sequence may be isolated from a suitable source, or it may be made synthetically or it may be prepared by use of recombinant DNA techniques.

Nucleotide Sequence

Aspects of the present invention may involve the use of nucleotide sequences, which are available in databases. These nucleotide sequences may be used to express amino acid sequences that may be used as a component of the composition of the present invention. In another embodiment, the nucleotide sequences may be used as a target to identify suitable agents for use in the composition of the present invention. In another embodiment, the nucleotide sequences may be used as a target to verify that an agent may be used as an inhibitor agent in the composition of the present invention.

As used herein, the term “nucleotide sequence” is synonymous with the term “polynucleotide”.

The nucleotide sequence may be DNA or RNA of genomic or synthetic or recombinant origin. The nucleotide sequence may be double-stranded or single-stranded whether representing the sense or antisense strand or combinations thereof.

The nucleotide sequence may be DNA.

The nucleotide sequence may be prepared by use of recombinant DNA techniques (e.g. recombinant DNA).

The nucleotide sequence may be cDNA.

The nucleotide sequence may be the same as the naturally occurring form, or may be derived therefrom.

Variants/Homologues/Derivatives

The present invention also encompasses the use of variants, homologues and derivatives of any thereof. Here, the term “homologue” means an entity having a certain homology with the subject amino acid sequences and the subject nucleotide sequences. Here, the term “homology” can be equated with “identity”.

In the present context, a homologous sequence is taken to include an amino acid sequence, which may be at least 75, 85 or 90% identical, preferably at least 95 or 98% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence, which may be at least 75, 85 or 90% identical, preferably at least 95 or 98% identical to the subject sequence. Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons may be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues, which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid#, 7-amino heptanoic acid*, L-methionine sulfone#*, L-norleucine*, L-norvaline*, p-nitro-L-phenylalanine*, L-hydroxyproline#, L-thioproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinoline-3-carboxyl acid)*, L-diaminopropionic acid# and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation involves the presence of one or more amino acid residues in peptoid form will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in to enhance the in vivo activity or life span of nucleotide sequences useful in the present invention.

The present invention may also involve the use of nucleotide sequences that are complementary to the sequences identified using the methods presented herein, or any derivative, fragment or derivative thereof. If the sequence is complementary to a fragment thereof then that sequence can be used as a probe to identify similar coding sequences in other organisms etc.

Hybridisation

The present invention may also encompass the use of nucleotide sequences that are capable of hybridising to the sequences mentioned herein, or any derivative, fragment or derivative thereof—such as if the agent is an anti-sense sequence.

The term “hybridization” as used herein shall include “the process by which a strand of nucleic acid joins with a complementary strand through base pairing” as well as the process of amplification as carried out in polymerase chain reaction (PCR) technologies.

The present invention may also encompass the use of nucleotide sequences that are capable of hybridising to the sequences that are complementary to the sequences identified using the methods presented herein, or any derivative, fragment or derivative thereof.

The term “variant” also encompasses sequences that are complementary to sequences that are capable of hydridising to other nucleotide sequences.

Preferably, the term “variant” encompasses sequences that are complementary to sequences that are capable of hydridising under stringent conditions (e.g. 50° C. and 0.2×SSC {1×SSC=0.15 M NaCl, 0.015 M Na₃citrate pH 7.0}) to nucleotide sequences.

More preferably, the term “variant” encompasses sequences that are complementary to sequences that are capable of hydridising under high stringent conditions (e.g. 65° C. and 0.1×SSC {1×SSC=0.15 M NaCl, 0.015 M Na₃citrate pH 7.0}) to nucleotide sequences.

Secretion

A polypeptide useful in the present invention may be secreted from the expression host into the culture medium from where the polypeptide may be more easily recovered.

Constructs

The term “construct”—which is synonymous with terms such as “conjugate”, “cassette” and “hybrid”—may include a nucleotide sequence useful in the present invention directly or indirectly attached to a promoter. The term “fused” includes direct or indirect attachment. In some cases, the terms do not cover the natural combination of the nucleotide sequence coding for the protein ordinarily associated with the wild type gene promoter and when they are both in their natural environment.

The construct may even contain or express a marker, which allows for the selection of the genetic construct in, for example, a bacterium, preferably of the genus Bacillus, such as Bacillus subtilis, or plants into which it has been transferred. Various markers exist which may be used, such as for example those encoding mannose-6-phosphate isomerase (especially for plants) or those markers that provide for antibiotic resistance—e.g. resistance to G418, hygromycin, bleomycin, kanamycin and gentamycin.

Vectors

The term “vector” includes expression vectors and transformation vectors and shuttle vectors.

The term “expression vector” means a construct capable of in vivo or in vitro expression.

The term “transformation vector” means a construct capable of being transferred from one entity to another entity—which may be of the species or may be of a different species. If the construct is capable of being transferred from one species to another—such as from an Escherichia coli plasmid to a bacterium, such as of the genus Bacillus, then the transformation vector is sometimes called a “shuttle vector”. It may even be a construct capable of being transferred from an E. coli plasmid to an Agrobacterium to a plant

Vectors may be transformed into a suitable host cell as described below to provide for expression of a polypeptide encompassed in the present invention. Thus, in a further aspect the invention provides a process for preparing polypeptides for use in the present invention which comprises cultivating a host cell transformed or transfected with an expression vector as described above under conditions to provide for expression by the vector of a coding sequence encoding the polypeptides, and recovering the expressed polypeptides.

The vectors may be for example, plasmid, virus or phage vectors provided with an origin of replication, optionally a promoter for the expression of the said polynucleotide and optionally a regulator of the promoter.

Vectors may contain one or more selectable marker genes. The most suitable selection systems for industrial micro-organisms are those formed by the group of selection markers which do not require a mutation in the host organism. Examples of fungal selection markers are the genes for acetamidase (amdS), ATP synthetase, subunit 9 (oliC), orotidine-5′-phosphate-decarboxylase (pvrA), phleomycin and benomyl resistance (benA). Examples of non-fungal selection markers are the bacterial G418 resistance gene (this may also be used in yeast, but not in filamentous fungi), the ampicillin resistance gene (E. coli), the neomycin resistance gene (Bacillus) and the E. coli uidA gene, coding for β-glucuronidase (GUS).

Vectors may be used in vitro, for example for the production of RNA or used to transfect or transform a host cell.

Thus, polynucleotides for use in the present invention may be incorporated into a recombinant vector (typically a replicable vector), for example a cloning or expression vector. The vector may be used to replicate the nucleic acid in a compatible host cell. Thus, quantities of polynucleotides may be made by introducing a polynucleotide into a replicable vector, introducing the vector into a compatible host cell, and growing the host cell under conditions, which bring about replication of the vector. The vector may be recovered from the host cell. Suitable host cells are described below in connection with expression vectors.

Genetically engineered host cells may be used to express an amino acid sequence (or variant, homologue, fragment or derivative thereof) in screening methods for the identification of agents and antagonists. Such genetically engineered host cells could be used to screen peptide libraries or organic molecules. Antagonists and agents such as antibodies, peptides or small organic molecules will provide the basis for pharmaceutical compositions. Such agents or antagonists may be administered alone or in combination with other therapeutics for the treatment of prion infection.

Expression Vectors

A nucleotide sequence may be incorporated into a recombinant replicable vector. The vector may be used to replicate and express the nucleotide sequence. Expression may be controlled using control sequences, which include promoters/enhancers and other expression regulation signals. Prokaryotic promoters and promoters functional in eukaryotic cells may be used. Tissue specific or stimuli specific promoters may be used. Chimeric promoters may also be used comprising sequence elements from two or more different promoters described above.

The protein produced by a host recombinant cell by expression of a nucleotide sequence may be secreted or may be contained intracellularly depending on the sequence and/or the vector used. The coding sequences can be designed with signal sequences, which direct secretion of the substance coding sequences through a particular prokaryotic or eukaryotic cell membrane.

Fusion Proteins

An amino acid sequence for use in the present invention may be produced as a fusion protein, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence to allow removal of fusion protein sequences. Preferably the fusion protein will not hinder the activity of the protein sequence.

The fusion protein may comprise an antigen or an antigenic determinant fused to the substance of interest. The fusion protein may be a non-naturally occurring fusion protein comprising a substance, which may act as an adjuvant in the sense of providing a generalised stimulation of the immune system. The antigen or antigenic determinant may be attached to either the amino or carboxy terminus of the substance.

An amino acid sequence may be ligated to a heterologous sequence to encode a fusion protein. For example, for screening of peptide libraries for agents capable of affecting the substance activity, it may be useful to encode a chimeric substance expressing a heterologous epitope that is recognized by a commercially available antibody.

Stereo and Geometric Isomers

The agents may exist as stereoisomers and/or geometric isomers—e.g. they may possess one or more asymmetric and/or geometric centres and so may exist in two or more stereoisomeric and/or geometric forms. The present invention contemplates the use of the entire individual stereoisomers and geometric isomers of those agents, and mixtures thereof. The terms used in the claims encompass these forms, provided said forms retain the appropriate functional activity (though not necessarily to the same degree).

Pharmaceutical Salt

The agent may be administered in the form of a pharmaceutically acceptable salt.

Pharmaceutically-acceptable salts are well known to those skilled in the art, and for example include those mentioned by Berge et al, in J. Pharm. Sci., 66, 1-19 (1977). Suitable acid addition salts are formed from acids which form non-toxic salts and include the hydrochloride, hydrobromide, hydroiodide, nitrate, sulphate, bisulphate, phosphate, hydrogenphosphate, acetate, trifluoroacetate, gluconate, lactate, salicylate, citrate, tartrate, ascorbate, succinate, maleate, fumarate, gluconate, formate, benzoate, methanesulphonate, ethanesulphonate, benzenesulphonate and p-toluenesulphonate salts.

When one or more acidic moieties are present, suitable pharmaceutically acceptable base addition salts can be formed from bases which form non-toxic salts and include the aluminium, calcium, lithium, magnesium, potassium, sodium, zinc, and pharmaceutically-active amines such as diethanolamine, salts.

A pharmaceutically acceptable salt of an agent may be readily prepared by mixing together solutions of an agent and the desired acid or base, as appropriate. The salt may precipitate from solution and be collected by filtration or may be recovered by evaporation of the solvent.

An agent may exist in polymorphic form.

An agent may contain one or more asymmetric carbon atoms and therefore exist in two or more stereoisomeric forms. Where an agent contains an alkenyl or alkenylene group, cis (E) and trans (Z) isomerism may also occur. The present invention includes the individual stereoisomers of an agent and, where appropriate, the individual tautomeric forms thereof, together with mixtures thereof.

Separation of diastereoisomers or cis- and tans-isomers may be achieved by conventional techniques, e.g. by fractional crystallisation, chromatography or H.P.L.C. of a stereoisomeric mixture of an agent or a suitable salt or derivative thereof. An individual enantiomer of an agent may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

The present invention also encompasses all suitable isotopic variations of an agent or a pharmaceutically acceptable salt thereof. An isotopic variation of an agent or a pharmaceutically acceptable salt thereof is defined as one in which at least one atom is replaced by an atom having the same atomic number but an atomic mass different from the atomic mass usually found in nature. Examples of isotopes that may be incorporated into an agent and pharmaceutically acceptable salts thereof include isotopes of hydrogen, carbon, nitrogen, oxygen, phosphorus, sulphur, fluorine and chlorine such as ²H, 3H, ¹³C, ¹⁴C, ¹⁵N, ¹⁷O, ¹⁸O, ³¹P, ³²P, ³⁵S, ¹⁸F and ³⁶Cl, respectively. Certain isotopic variations of an agent and pharmaceutically acceptable salts thereof, for example, those in which a radioactive isotope such as ³H or ¹⁴C is incorporated are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of an agent of the present invention and pharmaceutically acceptable salts thereof of this invention can generally be prepared by conventional procedures using appropriate isotopic variations of suitable reagents.

It will be appreciated by those skilled in the art that an agent may be derived from a prodrug. Examples of prodrugs include entities that have certain protected group(s) and which may not possess pharmacological activity as such, but may, in certain instances, be administered (such as orally or parenterally) and thereafter metabolised in the body to form an agent of the present invention which are pharmacologically active.

It will be further appreciated that certain moieties known as “pro-moieties”, for example as described in “Design of Prodrugs” by H. Bundgaard, Elsevier, 1985 (the disclosured of which is hereby incorporated by reference), may be placed on appropriate functionalities of agents. Such prodrugs are also included within the scope of the invention.

The present invention also includes the use of zwitterionic forms of an agent of the present invention. The terms used in the claims encompass one or more of the forms just mentioned.

Solvates

The present invention also includes the use of solvate forms of an agent of the present invention.

Pro-Drug

As indicated, the present invention may also include the use of pro-drug forms of an agent.

Pharmaceutically Active Salt

An agent may be administered as a pharmaceutically acceptable salt. Typically, a pharmaceutically acceptable salt may be readily prepared by using a desired acid or base, as appropriate. The salt may precipitate from solution and be collected by filtration or may be recovered by evaporation of the solvent.

Chemical Synthesis Methods

An agent may be prepared by chemical synthesis techniques. It will be apparent to those skilled in the art that sensitive functional groups may need to be protected and deprotected during synthesis of a compound of the invention. This may be achieved by conventional techniques, for example as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

It is possible during some of the reactions that any stereocentres present could, under certain conditions, be racemised, for example if a base is used in a reaction with a substrate having an optical centre comprising a base-sensitive group. This is possible during e.g. a guanylation step. It should be possible to circumvent potential problems such as this by choice of reaction sequence, conditions, reagents, protection/deprotection regimes, etc. as is well-known in the art

The compounds and salts of the invention may be separated and purified by conventional methods.

Separation of diastereomers may be achieved by conventional techniques, e.g. by fractional crystallisation, chromatography or H.P.L.C. of a stereoisomeric mixture of a compound of formula (I) or a suitable salt or derivative thereof. An individual enantiomer of a compound of formula (I) may also be prepared from a corresponding optically pure intermediate or by resolution, such as by H.P.L.C. of the corresponding racemate using a suitable chiral support or by fractional crystallisation of the diastereomeric salts formed by reaction of the corresponding racemate with a suitably optically active acid or base.

An agent or variants, homologues, derivatives, fragments or mimetics thereof may be produced using chemical methods to synthesize an agent in whole or in part. For example, if they are peptides, then peptides may be synthesized by solid phase techniques, cleaved from the resin, and purified by preparative high performance liquid chromatography (e.g., Creighton (1983) Proteins Structures And Molecular Principles, W H Freeman and Co, New York N.Y.). The composition of the synthetic peptides may be confirmed by amino acid analysis or sequencing (e.g., the Edman degradation procedure; Creighton, supra).

Synthesis of peptide agents may be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences comprising an agent or any part thereof may be altered during direct synthesis and/or combined using chemical methods with a sequence from other subunits, or any part thereof, to produce a variant agent.

In an alternative embodiment of the invention, the coding sequence of a peptide agent (or variants, homologues, derivatives, fragments or mimetics thereof) may be synthesized, in whole or in part, using chemical methods well known in the art (see Caruthers M H et al (1980) Nuc Acids Res Symp Ser 215-23, Horn T et al (1980) Nuc Acids Res Symp Ser 225-232).

Mimetic

As used herein, the term “mimetic” relates to any chemical which includes, but is not limited to, a peptide, polypeptide, antibody or other organic chemical which has the same qualitative activity or effect as a reference agent.

Chemical Derivative

The term “derivative” or “derivatised” as used herein includes chemical modification of an agent. Illustrative of such chemical modifications would be replacement of hydrogen by a halo group, an alkyl group, an acyl group or an amino group.

Chemical Modification

The chemical modification of an agent may either enhance or reduce hydrogen bonding interaction, charge interaction, hydrophobic interaction, Van Der Waals interaction or dipole interaction between the agent and the target.

In one aspect, the identified agent may act as a model (for example, a template) for the development of other compounds.

Recombinant Methods

An agent or target may be prepared by recombinant DNA techniques.

Other Active Components

A composition may comprise other therapeutic substances in addition to the agent.

Antibody

An agent for use in the composition may comprise one or more antibodies.

The “antibody” as used herein includes but is not limited to, polyclonal, monoclonal, chimeric, single chain, Fab fragments and fragments produced by a Fab expression library. Such fragments include fragments of whole antibodies which retain their binding activity for a target substance, Fv, F(ab′) and F(ab′)2 fragments, as well as single chain antibodies (scFv), fusion proteins and other synthetic proteins which comprise the antigen-binding site of the antibody. Furthermore, the antibodies and fragments thereof may be humanised antibodies, for example as described in U.S. Pat. No. 239,400. Neutralizing antibodies, i.e., those, which inhibit biological activity of the substance polypeptides, are especially preferred for diagnostics and therapeutics.

Antibodies may be produced by standard techniques, such as by immunisation with the substance of the invention or by using a phage display library.

If polyclonal antibodies are desired, a selected mammal (e.g., mouse, rabbit, goat, horse, etc.) is immunised with an immunogenic polypeptide bearing epitope(s) obtainable from an identified agent and/or substance of the present invention. Depending on the host species, various adjuvants may be used to increase immunological response. Such adjuvants include, but are not limited to, Freund's, mineral gels such as aluminium hydroxide, and surface-active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance polypeptide is administered to immunologically compromised individuals for the purpose of stimulating systemic defence.

Serum from the immunised animal is collected and treated according to known procedures. If serum containing polyclonal antibodies to an epitope obtainable from an identified agent and/or substance of the present invention contains antibodies to other antigens, the polyclonal antibodies may be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against particular epitopes may also be readily produced by one skilled in the art. The general methodology for making monoclonal antibodies by hybridomas is well known. Immortal antibody-producing cell lines may be created by cell fusion, and also by other techniques such as direct transformation of B lymphocytes with oncogenic DNA, or transfection with Epstein-Barr virus. Panels of monoclonal antibodies produced against orbit epitopes may be screened for various properties; i.e., for isotype and epitope affinity.

Monoclonal antibodies may be prepared using any technique, which provides for the production of antibody molecules by continuous cell lines in culture. These include, but are not limited to, the hybridoma technique originally described by Koehler and Milstein (1975 Nature 256:495-497), the human B-cell hybridoma technique (Kosbor et al (1983) Immunol Today 4:72; Cote et al (1983) Proc Natl Acad Sci 80:2026-2030) and the EBV-hybridoma technique (Cole et al (1985) Monoclonal Antibodies and Cancer Therapy, Alan R Liss Inc, pp 77-96). In addition, techniques developed for the production of “chimeric antibodies”, the splicing of mouse antibody genes to human antibody genes to obtain a molecule with appropriate antigen specificity and biological activity may be used (Morrison et al (1984) Proc Natl Acad Sci 81:6851-6855; Neuberger et al (1984) Nature 312:604-608; Takeda et al (1985) Nature 314:452-454). Alternatively, techniques described for the production of single chain antibodies (U.S. Pat. No. 4,946,779) may be adapted to produce the substance specific single chain antibodies.

Antibodies may also be produced by inducing in vivo production in the lymphocyte population or by screening recombinant immunoglobulin libraries or panels of highly specific binding reagents as disclosed in Orlandi et al (1989, Proc Natl Acad Sci 86: 3833-3837), and Winter G and Milstein C (1991; Nature 349:293-299).

Antibody fragments which contain specific binding sites for the substance may also be generated. For example, such fragments include, but are not limited to, the F(ab′)2 fragments which may be produced by pepsin digestion of the antibody molecule and the Fab fragments which may be generated by reducing the disulfide bridges of the F(ab′)2 fragments. Alternatively, Fab expression libraries may be constructed to allow rapid and easy identification of monoclonal Fab fragments with the desired specificity (Huse W D et al (1989) Science 256:1275-128 1).

General Assay Techniques

Any one or more of appropriate targets—such as an amino acid sequence and/or nucleotide sequence of a prion susceptibility protein or gene—may be used for identifying an agent according to the present invention.

The target employed in such a test may be free in solution, affixed to a solid support borne on a cell surface, or located intracellularly. The abolition of target activity or the formation of binding complexes between the target and the agent being tested may be measured.

The method of the present invention may be a screen, whereby a number of agents are tested for modulating prion infection.

Techniques for drug screening may be based on the method described in Geysen, European Patent Application 84/03564, published on Sep. 13, 1984. In summary, large numbers of different small peptide test compounds are synthesized on a solid substrate, such as plastic pins or some other surface. The peptide test compounds are reacted with a suitable target or fragment thereof and washed. Bound entities are then detected—such as by appropriately adapting methods well known in the art. A purified target may also be coated directly onto plates for use in a drug screening techniques. Alternatively, non-neutralising antibodies may be used to capture the peptide and immobilise it on a solid support.

It is expected that the methods of the present invention will be suitable for both small and large-scale screening of test compounds as well as in quantitative assays.

In one preferred aspect, the present invention relates to a method of identifying agents capable of modulating the prion infection.

Reporters

A wide variety of reporters may be used to screen for agents identified in the method of the present invention with preferred reporters providing conveniently detectable signals (eg. by spectroscopy). By way of example, a number of companies such as Pharmacia Biotech (Piscataway, N.J.), Promega (Madison, Wis.), and US Biochemical Corp (Cleveland, Ohio) supply commercial kits and protocols for assay procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. NO. 4,366,241.

Host Cells

The term “host cell” may include any cell that could comprise the target for the agent of the present invention.

Thus, a further embodiment of the present invention provides host cells transformed or transfected with a polynucleotide that is or expresses the target of the present invention. Preferably said polynucleotide is carried in a vector for the replication and expression of polynucleotides that are to be the target or are to express the target. The cells will be chosen to be compatible with the said vector and may for example be prokaryotic (for example bacterial), fungal, yeast or plant cells.

The gram-negative bacterium E. coil is widely used as a host for heterologous gene expression. However, large amounts of heterologous protein tend to accumulate inside the cell. Subsequent purification of the desired protein from the bulk of E. coil intracellular proteins can sometimes be difficult

In contrast to E. coli, bacteria from the genus Bacillus are very suitable as heterologous hosts because of their capability to secrete proteins into the culture medium. Other bacteria suitable as hosts are those from the genera Streptomyces and Pseudomonas.

Depending on the nature of the polynucleotide encoding the polypeptide useful in the present invention, and/or the desirability for further processing of the expressed protein, eukaryotic hosts such as yeasts or other fungi may be preferred. In general, yeast cells are preferred over fungal cells because they are easier to manipulate. However, some proteins are either poorly secreted from the yeast cell, or in some cases are not processed properly (e.g. hyperglycosylation in yeast). In these instances, a different fungal host organism should be selected.

Examples of suitable expression hosts within the scope of the present invention are fungi such as Aspergillus species (such as those described in EP-A-0184438 and EP-A-0284603) and Trichoderma species; bacteria such as Bacillus species (such as those described in EP-A-0134048 and EP-A-0253455), Streptomyces species and Pseudomonas species; and yeasts such as Kluyveromyces species (such as those described in EP-A-0096430 and EP-A-0301670) and Saccharomyces species. By way of example, typical expression hosts may be selected from Aspergillus niger, Aspergillus niger var. tubigenis, Aspergillus niger var. awamori, Aspergillus aculeatis, Aspergillus nidulans, Aspergillus orvzae, Trichoderma reesei, Bacillus subtilis, Bacillus licheniformis, Bacillus amyloliquefaciens, Kluyveromyces lactis and Saccharomyces cerevisiae.

The use of suitable host cells—such as yeast, fungal and plant host cells—may provide for post-translational modifications (e.g. myristoylation, glycosylation, truncation, lapidation and tyrosine, serine or threonine phosphorylation) as may be needed to confer optimal biological activity on recombinant expression products of the present invention.

Organism

The term “organism” includes any organism that could comprise the target according to the present invention and/or products obtained therefrom. Examples of organisms may include a fungus, yeast or a plant.

The term “transgenic organism” in relation to the present invention includes any organism that comprises the target according to the present invention and/or products obtained.

Therapy

Agents identified by the method of the present invention may be used as therapeutic agents—i.e. in therapy applications.

As with the term “treatment”, the term “therapy” includes curative effects, alleviation effects, and prophylactic effects.

The therapy may be on mammals such as humans or livestock.

The therapy may be for treating conditions associated with prion infection.

Pharmaceutical Compositions

Pharmaceutical compositions useful in the present invention may comprise a therapeutically effective amount of agent(s) and pharmaceutically acceptable carrier, diluent or excipient (including combinations thereof).

Pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent may be selected with regard to the intended route of administration and standard pharmaceutical practice. Pharmaceutical compositions may comprise as—or in addition to—the carrier, excipient or diluent any suitable binder(s), lubricant(s), suspending agent(s), coating agent(s) or solubilising agent(s).

Preservatives, stabilizers, dyes and even flavoring agents may be provided in pharmaceutical compositions. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, pharmaceutical compositions useful in the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

Agents may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

If an agent is a protein, then said protein may be prepared in situ in the subject being treated. In this respect, nucleotide sequences encoding said protein may be delivered by use of non-viral techniques (e.g. by use of liposomes) and/or viral techniques (e.g. by use of retroviral vectors) such that the said protein is expressed from said nucleotide sequence.

Administration

The term “administered” includes delivery by viral or non-viral techniques. Viral delivery mechanisms include but are not limited to adenoviral vectors, adeno-associated viral (AAV) vectos, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

The components useful in the present invention may be administered alone but will generally be administered as a pharmaceutical composition—e.g. when the components are in admixture with a suitable pharmaceutical excipient, diluent or carrier selected with regard to the intended route of administration and standard pharmaceutical practice.

For example, the components may be administered (e.g. orally) in the form of tablets, capsules, ovules, elixirs, solutions or suspensions, which may contain flavouring or colouring agents, for immediate-, delayed-, modified-, sustained-, pulsed- or controlled-release applications.

If the pharmaceutical is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

It is to be understood that not all of the components of the pharmaceutical need be administered by the same route. Likewise, if the composition comprises more than one active component, then those components may be administered by different routes.

If a component is administered parenterally, then examples of such administration include one or more of: intravenously, intra-arterially, intraperitoneally, intrathecally, intraventricularly, intraurethrally, intrasternally, intracranially, intramuscularly or subcutaneously administering the component; and/or by using infusion techniques.

For parenteral administration, the component is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the component(s) useful in the present invention may be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the component(s) may be administered in the form of a suppository or pessary, or it may be applied topically in the form of a gel, hydrogel, lotion, solution, cream, ointment or dusting powder. The component(s) may also be dermally or transdermally administered, for example, by the use of a skin patch. They may also be administered by the pulmonary or rectal routes. They may also be administered by the ocular route. For ophthalmic use, the compounds may be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the component(s) may be formulated as. a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water.

Alternatively, it may be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, diet, mode and time of administration, rate of excretion, drug combination, the severity of the particular condition, and the individual undergoing therapy.

Formulation

The component(s) may be formulated into a pharmaceutical composition, such as by mixing with one or more of a suitable carrier, diluent or excipient, by using techniques that are known in the art.

Animal Test Models

In vivo models may be used to investigate and/or design therapies or therapeutic agents to modulate prion infection. The models could be used to investigate the effect of various tools/lead compounds on a variety of parameters, which are implicated in the development of or treatment of prion infection. These animal test models may be used as, or in, the method of the present invention. The animal test model will be a non-human animal test model.

General Recombinant DNA Methodology Techniques

Although in general the techniques mentioned herein are well known in the art, reference may be made in particular to Sambrook et al., Molecular Cloning, A Laboratory Manual (1989) and Ausubel et al., Short Protocols in Molecular Biology (1999) 4^{th} Ed, John Wiley & Sons, Inc. PCR is described in U.S. Pat. No. 4,683,195, U.S. Pat. No. 4,800,195 and U.S. Pat. No. 4,965,188.

In another aspect of the present invention, a secondary passage of prions in SJL mice may be used as a confirmatory test for prion infection. As used herein, the term “passage” refers to the method of contacting SJL mice with a sample from a prion infected SJL mouse, said prion infected SJL mouse having previously been contacted with a sample and showing symptoms of prion infection. Preferably, the prion incubation time following the second passage is shorter than the first passage. Preferably, the attack rate from the second passage will be higher than the first passage.

In another aspect of the present invention, use of reference stain(s) of test animals may augment the methods of the present invention. As used herein, the term “reference strain” refers to a test animal that is known to be susceptible to prion infection from a given species such as a bovine or human. Accordingly, a sample from an unknown origin is contacted with SJL mice, which subsequently show symptoms of prion infection. By contacting the same sample with a range of reference strains that are each only susceptible to prions from certain host(s) then the source of the prion may advantageously be established. Such reference strains may be mice transgenic for Prion Protein Modulator Factor (PPMF). PPMF is species specific and is able to bind to PrP^{C}, causing a conformational change from PrP^{C} into PrP^{Sc}. PPMF is disclosed in U.S. Pat. No. 5,962,669.

### EXAMPLES

The present invention is illustrated with reference to the following examples.

### Example 1

Detection of Prions in a Sample

Test animals are contacted with the sample as follows.

Sample preparation is performed in a microbiological containment level III facility. Samples are derived from the frozen brain tissue of a cow that is to be tested for the presence of prions. The sample is dissociated using a sterile disposable homogeniser in phosphate buffered saline. The suspension is then subjected to repeated extrusion through an 18-gauge syringe needle followed by a 22-gauge needle. Samples for inoculation into SJL mice are diluted in PBS to prepare a 1% (w/v) brain extract.

The SJL mice to be contacted with the sample to be tested are bred in an animal microbiological containment level I facility and identified by ear punching or by transponder tagging. Prior to contact with the sample, the SJL mice are anaethetised with halothane/O₂. Forty SJL mice are each contacted with 30 μl of 1% (w/v) brain extract via intra-cerebral injection into the right parietal lobe of the brain using a 27-gauge needle. The procedure is performed in a containment level III facility.

Following contact with the sample, the SJL mice are incubated with the sample in an animal microbiological containment level III facility.

All the SJL mice are monitored for adverse effects every 3 days. If clinical signs of prion infection appear, the mice are examined daily and culled if showing signs of distress by CO₂ asphyxiation. Criteria for clinical diagnosis of prion infection in mice have been described by Carlson et al. (1986), Cell 46, 503-511. The prion incubation time is also measured. This is done by noting the amount of time that elapses from contacting each of the test animals with the sample, to the time when the test animals first display adverse effects or death resulting from prion infection. Twenty five out of the 40 test animals showed symptoms of prion infection 196±13 days after contact with the sample.

Biopsies are performed to confirm prion infection as follows.

Prion infection in the 25 mice is confirmed using Western blot analysis using antibodies raised against BSE prions. 10% (w/v) brain homogenates are prepared in cold lysis buffer (10 mM Tris-HCl and 10 mM EDTA, pH 7.4, 100 mM NaCl, 0.5% NP-40, 0.5% sodium deoxycholate in PBS). Insoluble material is removed by centrifugation at 3000 rpm for 5 minutes. Proteinase K digestion (50 mg/ml) is performed for 1 hour at 37° C. The reaction is terminated by the addition of Pefabloc (Boehringer) to a final concentration of 2 mM. Samples are boiled for 5 minutes in an equal volume of loading buffer (125 mM Tris-HCl, pH 6.8, 20% glycerol, 4% SDS, 0.02% bromophenol blue) before electrophoresis on 16% tris-glycine gels. Gels are blotted onto Immobilon-P membranes, blocked in 5% Blotto (5% non-fat milk powder in PBS with 0.05% Tween-20) followed by incubation overnight with the monoclonal antibody ICSM18. Blots are washed in PBS, 0.05% Tween-20, and incubated With an alkaline-phosphatase conjugated anti-mouse antibody for 1 hour at room temperature. Blots are washed again and developed with a chemifluorescent substrate (Amersham) and visualised on a Storm 840 phosphoimager (Molecular Dynamics).

Thus it is demonstrated that prions have been detected in the sample.

### Example 2

Primary Passage of BSE Prions to Mice

Prions are detected in a sample according to the methods of Example 1 except that the sample is derived from the frozen brain tissue of a cow in which clinical diagnosis of BSE has previously been confirmed by histopathological examination. This is done by removing the brains and immersion-fixing in 10% buffered formalin. The brain is then embedded in paraffin and 7 μM thick sections are cut. Serial sections are stained with hematoxylin, eosin and Gomori trichrome.

Seven different strains of mice are contacted intra-cerebrally with the sample and the prion incubation time in each strain is measured. Results are shown in Table 1.

TABLE 1

| Strain of mice | Mean incubation time ± s.e.m^{a} | Attack rate^{b} |
|---|---|---|
| C57BL6/OlaHsd | 710 ± 15 (n = 6) | 21% (n = 29) |
| FVB/NHsd | 589 ± 21 (n = 22) | 71% (n = 31) |
| NZW/OlaHsd | 555 ± 28 (n = 6) | 35% (n = 17) |
| RIIIS/J | 241 ± 14 (n = 20) | 74% (n = 27) |
| SJL/OlaHsd | 196 ± 13 (n = 25) | 63% (n = 40) |
| SM/J | 273 ± 34 (n = 11) | 48% (n = 23) |
| SWR/OlaHsd | 640 ± 26 (n = 11) | 55% (n = 20) |

^{a}n = number of test animals that display symptons of prion infection.
^{b}n = total number of test animals used in each experiment.

When SJL mice are contacted with a sample containing prions that cause BSE in their appropriate host, there is a substantial reduction in prion incubation time to 196±13 days compared to the six other strains of mice tested (range is 241+15 days to 710+15 days).

Thus it is demonstrated that SJL mice have advantageously short prion incubation times.

### Example 3

Secondary Passage of BSE to Mice

The experiment is performed according to Example 2 except that the sample is derived from the brain tissue of SJL mice that have been passaged once with a sample containing prions that cause BSE in their appropriate host. Furthermore, the sample is contacted with SJL mice and FVB mice.

The results are shown in Table 2.

TABLE 2

| | | Mean incubation | |
|---|---|---|---|
| Inoculum | Mouse strain | time ± s.e.m | Attack rate |
| BSE to SJL (I1590) | SJL | 125 ± 3 (n = 7) | 100% (n = 7) |
| BSE to SJL (I1590) | FVB | 135 ± 3 (n = 9) | 75% (n = 12) |

Secondary passage of BSE in SJL mice results in a decrease in incubation time from 196±13 days (see Example 2) to 125±3 days. AU SJL mice (7/7) develop clinical disease and so the attack rate is 100%. The prion incubation time is shorter and the attack rate is higher in SJL mice than it is in FVB mice. The presence of BSE prions in the SJL and FVB mice is confirmed by Western blotting.

Thus it is demonstrated that secondary passage of BSE to SJL mice results in a shorter prion incubation time and an increase in attack rate.

### Example 4

Primary Passage of CJD and vCJD to SJL Mice

Prions are detected in samples according to the methods in Example I with the exception that the samples are derived from the frozen brain tissue of humans in which clinical diagnosis of CJD or vCJD is confirmed by histopathological examination. Primary passage of CJD and vCJD into SJL mice is performed and prion incubation times determined.

Results are shown in Table 3.

TABLE 3

| Inoculum | Mean incubation time ± s.e.m | | Attack rate |
|---|---|---|---|
| vCJD (I336) | 139 ± 17 | (n = 6) | 60% (n = 10) |
| vCJD (I344) | 256 ± 46 | (n = 5) | 50% (n = 10) |
| vCJD (I342) | 169, 169 | (n = 2) | 18% (n = 11) |
| CJD (I 1197) | 208 ± 17 | (n = 2) | 29% (n = 7) |

| | | |
|---|---|---|
| CJD (I 026) | (n = 0) | 0% (n = 8) |
| CJD (I 1477) | (n = 0) | 0% (n = 8) |

SJL mice inoculated with vCJD have prion incubation times that are substantially shorter than those for CJD. This suprising finding suggests that SJL mice are more susceptible to prions that cause vCJD than CJD. The mean incubation time (across the different vCJD inocula) is 189d±24.

Furthermore, these results also illustrate the suprising finding that that SJL mice are more susceptible to prions that cause BSE (196±13 days, Table 1) than CJD.

Thus it is demonstrated that SJL mice contacted with prions that cause vCJD in their appropriate host have prion incubation times of 189 days or less.

### Example 5

Identification of Prion Susceptibility Genes

Prion incubation times are measured according to Example 2 except that SJL and C57B16 mice are used. SJL mice have been shown to have prion incubation times of 196±13 days following primary passage with prions that cause BSE in their appropriate host and are therefore considered to be susceptible mice. C57B16 mice have prion incubation times of 710±15 days following primary passage with prions that cause BSE in their appropriate host and are therefore considered to be non-susceptible mice.

cDNA is prepared from the mRNA of a SJL mouse (first mRNA) and C57B16 mouse (second mRNA). 50 ng-1 μg of mRNA is mixed with 500 ng of an oligo (dT) primer and the mixture is heated to 70° C. for 10 minutes and immediately cooled on ice. 10 mM DNTP mix, 100 mM DTT, reverse transcriptase buffer and reverse transcriptase are then added. The reaction is incubated at 42° C. for 90 minutes and then terminated with 60 mM EDTA. The two sets of cDNA are hybridised to each other two or three times in succession to a 20-fold excess of the second mRNA and the cDNA:mRNA hybrids are removed by chromatography on hydroxyapatite. The unhybridised cDNA from the SJL mouse is annealed to a 100-fold excess of the mRNA preparation from which it was originally synthesised, and the resulting cDNA:mRNA hybrids are recovered by chromatography on hydroxyapatite. Once the mRNA is removed by alkaline hydrolysis, the cDNA, which is highly enriched for sequences specific to the mRNA of the SJL mouse, is used to probe a cDNA library for clones homologous to these sequences. The resulting clones are candidate prion susceptibility genes or fragments thereof

Thus it is demonstrated that prion susceptibility genes are identified by comparing genes of susceptible and non-suspectible test animals.

### Example 6

Identification of Agents that Increase or Decrease Prion Incubation Times.

Prion detection is performed according to Example 1 with the exception that the sample is derived from the frozen brain tissue of a human in which clinical diagnosis of vCJD has previously been confirmed by histopathological examination. Twenty SJL mice are contacted with the sample. Another 20 SJL mice are also contacted with the same sample in addition to an agent to be tested. The agent is administered separately and immediately after contact with the sample. The agent is administered in a volume of 30 μl intra-cerebrally using a 27-gauge needle into the right parietal lobe of the brain.

Clearly, it may be advatageous to carry out this Example by administering an agent before or at the time of prion inoculation, or to inoculate first and administer an agent during course of infection (preferably before symptoms of disease).

Thus it is demonstrated that an agent is identified that increases or decreases the prion incubation time.

### Example 7

Estimating the Amount of Prions in a Sample.

Prion detection is performed according to Example 1 using a sample derived from a cow in which clinical diagnosis of BSE has previously been confirmed by histopathological examination.

The test animals used are SJL mice. When the SJL mice are monitored, the amount of time taken for the SJL mice to display symptoms of prion infection and the amount of time taken for the SJL mice to die is noted. The time interval between the two parameters is then calculated.

A calibration curve can be used to estimate BSE titers from time intervals.

The calibration curve is produced by contacting SJL mice with samples at different dilutions containing known titers of BSE prions. The time intervals from onset of illness to death are measured for each dilution. A calibration curve of time intervals from first manifestation of symptoms to death versus prion titre is plotted.

SJL mice are then contacted with the test sample at different dilutions. For each dilution, four SJL mice are inoculated. Dilutions in the range of 10³ to 10⁹ ID₅₀ Units/ml are used. The mice are then incubated and the time intervals from onset of illness to death are measured for each mouse at each dilution, and averaged per dilution if appropriate.

The amount of prions in the sample is estimated by reading from the calibration curve.

Thus it is demonstrated that the methods of the present invention may be used to estimate the amount of prions in a sample.

### Example 8

Estimating the Susceptibility of Test Animals to Prion Infection.

Prion detection is performed according to Example I except that the samples are derived from the frozen brain tissue of a cow in which clinical diagnosis of BSE is confirmed by histopathological examination. The mice used are the same as those used in Example 2. PrP glycoforms are quantified by incubating the Western blots (after washing) with a horseradish peroxidase-conjugated anti-mouse antibody for 1 hour. The blot is then washed again and developed using a chemiluminescent substrate (ECL, Amersham) and Biomax MR film (Kodak).

The results of this experiment show that BSE prions passaged in FVB, Tg152, SWR, NZW, C57BL6 and SM/J mice give a characteristic glycoform ratio where the di-glycosylated band is stained more intensely. In SJL mice BSE prions show an altered glycoform ratio where the mono-glycosylated band appears dominant. This is also seen in RIIIS/J mice. With this information, it is estimated that the prion incubation times will be shortest in SJL and RIIIS/J mice i.e. that SJL and RIIIS/J are most susceptible to prion infection.

This is tested by determining the prion incubation times in all seven strains following primary passage with BSE. The results are shown in Table 4.

TABLE 4

| Strain of mice | Mean incubation time ± s.e.m^{a} | Attack rate^{b} |
|---|---|---|
| C57BL6/OlaHsd | 710 ± 15 (n = 6) | 21% (n = 29) |
| FVB/NHsd | 589 ± 21 (n = 22) | 71% (n = 31) |
| NZW/OlaHsd | 555 ± 28 (n = 6) | 35% (n = 17) |
| RIIIS/J | 241 ± 14 (n = 20) | 74% (n = 27) |
| SJL/OlaHsd | 196 ± 13 (n = 25) | 63% (n = 40) |
| SM/J | 273 ± 34 (n = 11) | 48% (n = 23) |
| SWR/OlaHsd | 640 ± 26 (n = 11) | 55% (n = 20) |

^{a}n = number of test animals that display symptons of prion infection.
^{b}n = total number of test animals used in each experiment.

The results confirm that SJL and RIIIS/J mice have the shortest prion incubation times.

Thus it is demonstrated that the susceptibility of test animals to prion infection is estimated using glycoform ratio analysis.

### Example 9

Third Passage of BSE to Mice

The experiment is performed according to Example 2 except that the sample is derived from the brain tissue of SJL mice that have been passaged twice with a sample containing prions that cause BSE in their appropriate host. The sample is contacted with SJL mice and FVB mice.

| | Mouse | Mean incubation | |
|---|---|---|---|
| Inoculum | strain | time ± s.e.m | Attack rate |
| BSE→SJL→SJL (I 1891) | SJL | 110 ± 3 (n = 8) | 100% (n = 8) |
| BSE→SJL→SJL (I 1891) | FVB | 148 + 4 (n = 5) | 83% (n = 6) |

On third passage of BSE to SJL mice the incubation time is further reduced from 125±3 days to 110±3 days. All mice develop clinical disease and so the attack rate is 100%. The prion incubation time is shorter and the attack rate is higher in SJL mice than it is in FVB mice. The presence of BSE prions in the SJL and FVB mice is confirmed by Western blotting.

The experiment is performed according to example 2 except that the sample is derived from the brain tissue of clinically sick FVB mice infected with SJL passaged BSE. The sample is contacted with SJL and FVB mice.

| | Mouse | Mean incubation | |
|---|---|---|---|
| Inoculum | strain | time ± s.e.m | Attack rate |
| BSE→SJL→FVB (I2472) | SJL | 114 ± 3 (n = 9) | 90% (n = 10) |
| BSE→SJL→FVB (I2472) | FVB | 132 ± 5 (n = 10) | 100% (n = 10) |

### Example 10

Second Passage of vCJD

The experiment is performed according to example 2 except that the sample is derived from the brain tissue of SJL mice that have been passaged once with a sample containing prions that cause vCJD in their appropriate host.

| | Mouse | Mean incubation | | |
|---|---|---|---|---|
| Inoculum | strain | time ± s.e.m | | Attack rate |
| vCJD→SJL (I1892) | SJL | 127 ± 1 | (n = 9) | 100% (n = 9) |
| vCJD→SJL (I1892) | FVB | 151, 149 | (n = 2) | 33% (n = 6) |
| vCJD→SJL (I2362) | SJL | 132 ± 2 | (n = 10) | 100% (n = 10) |
| vCJD→SJL (I2362) | FVB | 159 ± 4 | (n = 8) | 100% (n = 8) |
| vCJD→SJL (I2354) | SJL | 138 ± 4 | (n = 7) | 78% (n = 9) |
| vCJD→SJL (I2354) | FVB | 160 ± 3 | (n = 9) | 90% (n = 10) |

Secondary passage of vCJD in SJL mice results in a decrease in incubation time (see also example 4). Most SJL mice develop clinical signs of disease before death and this is reflected in the increased attack rate (see also example 4).

SJL Mice References

Angel C. R., Mahin D. T., Farris R. D., and Woodward K. T. (1967) Heritability of plasma cholinesterase activity in inbred mouse strains. Science 156, 529-530.

Barthold S. W., Beck D. S., Hansen G. M., Terwilliger G. A., and Moody K. D. (1990) Lyme borreliosis in selected strains and ages of laboratory mice. J. Infect. Dis. 162, 133-138.

Beamer W. G., Donahue L. R., Rosen C. J., and Baylink D. J. (1996) Genetic-variability in adult bone-density among inbred strains of mice. Bone 18, 397-403.

Bebo B. F., Lee C. H., Orr E. L., and Linthicum D. S. (1996) Mast cell-derived histamine and tumor-necrosis-factor—differences between SJL/J and BALB/c inbred strains of mice. Immunology and Cell Biology 74, 225-230.

Belknap J. K., Crabbe J. C., Riggan J., and O'Toole L. A. (1993) Voluntary consumption of morphine in 15 inbred mouse strains. Psychopharmacology 112, 352-358.

Beutner U., Launois P., Ohteki T., Louis J. A., and MacDonald H. R. (1997) Natural killer-like T cells develop in SJL mice despite genetically distinct defects in NK1.1 expression and in inducible interleukin-4 production. Eur. J. Immunol. 27, 928-934.

Bhathal P. S., Jordan T. W., and Mackay I. R. (1990) Mouse strain differences in susceptibility to sporidesmin-induced biliary tract injury. Liver 10, 193-204.

Bittner, R. E.; Anderson, L. V. B.; Burkhardt, E.; Bashir, R.; Vafiadaki, E.; Ivanova, S.; Raffelsberger, T.; Maerk, I.; Hoger, H.; Jung, M.; Karbasiyan, M.; Storch, M.; Lassmann, H.; Moss, J. A.; Davison, K.; Harrison, R.; Bushby, K. M. D.; Beis, A (1999). Dysferlin deletion in SJL mice (SJL-Dysf) defines a natural model for limb girdle muscular dystrophy 2B. (Letter) Nature Genet. 23: 141-142.

Blizard D. A. and Welty R. (1971) Cardiac activity in the mouse: strain differences. J. Comp. Physiol. Psychol. 77, 337-344.

Blomberg B., Geckeler W. R., and Weigert M. (1972) Genetics of the antibody response to Dextran in mice. Science 177, 178-180.

Borel Y. and Kilham L. (1974) Carrier-determined tolerance in various strains of mice: the role of isogenic IgG in the induction of hapten specific tolerance. Proc. Soc. Exp. Biol. Med. 145, 470-474.

Boucher D. W., Hayashi K., Rosenthal J., and Notkins A. L. (1975) Virus-induced diabetes mellitus. III. Influence of sex and strain of host. J. Infect. Dis. 131, 462-466.

Brilliant M. H., Ching A., Nakatsu Y., and Eicher E. M. (1994) The original pink-eyed dilution mutation (p) arose in asiatic mice: Implications for the H4 minor histocompatibility antigen, Myod1 regulation and the origin of inbred strains. Genetics 138, 203-211.

Caffe A. R., Szel A., Juliusson B., Hawkins R., and vanVeen T. (1993) Hyperplastic neuroretinopathy and disorder of pigment epithelial cells precede accelerated retinal degeneration in the SJL/N mouse. Cell & Tissue Research 271, 297-307.

Clerici E. (1972) Induction of amyloidosis in mice upon treatment with polypeptidylcaseins and DNP-casein. Relation to antigenicity. Acta Pathol. Microbiol. Scand. Suppl., 233, 167-171.

Cooper P. A., Benno R. H., Hahn M. E., and Hewitt J. K. (1991) Genetic analysis of cerebellar foliation patterns in mice (Mus musculus). Behav. Genet. 21, 405-419.

Crispens C. G. (1973) Some characteristics of strain SJL/JDg mice. Lab. Animal Sci. 23, 408-413.

Dagg C. P. (1966) Teratogenesis, in Biology of the laboratory mouse, 2nd. ed. (Green E. L., ed), pp.309-328. McGraw-Hill, New York.

De Souza C. M., Maia L. C. S., and Vaz N. M. (1974) Susceptibility to cutaneous anaphylaxis in inbred strains of mice. J. Immunol. 112, 1369-1372.

Dietz M. and Rick M. A. (1972) Effect of host strain and H-2 type on spontaneous regression of murine leukemia virus. Int. J. Cancer. 10, 99-104.

Dorf M. E., Dunham E. K., Johnson J. P., and Benacerraf B. (1974) Genetic control of the immune response: the effect of non-H-2 linked genes on antibody production. J. Immunol. 112, 1329-1336.

Festing, M. F. W. (1979). Inbred strains in biomedica research. New York: Oxford University Press. 255.

Fuchs S., Mozes E., Maoz A., and Sela M. (1974) Thymus independence of a collagen-like synthetic polypeptide and of collagen, and the need for thymus and bone marrow-cell cooperation in the immune response to gelatin. J. Exp. Med. 139, 148-158.

Fujinaga S., Poel W. E., Williams W. C., and Dmochowski L. (1970) Biological and morphological studies of SJL/J strain reticulum cell neoplasms induced and transmitted serially in low leukemia-strain mice. Cancer Res. 30, 729-742.

Fujiwara M. and Cinader B. (1974) Cellular aspects of tolerance. IV. Strain variations of tolerance inducibility. Cell. Immunol. 12, 11-29.

Gross S. and Hutton J. (1971) Induction of hepatic -aminolaevulinic acid synthetase activity in strains of inbred mice. J. Biol. Chem. 246, 606-614.

Heiniger H. J., Taylor B. A., Hards E. J., and Meier H. (1975) Heritability of the phytohaemagglutinin responsiveness of lymphocytes and its relationship to leukemogenesis. Cancer Res. 35, 825-831.

Hill G. B., Osterhout S., and O'Fallon W. M. (1968) Variation in response to hyperbaric oxygen among inbred strains of mice. Proc. Soc. Exp. Biol. Med. 129, 687-689.

Hudson S. J., Dix R. D., and Streilein J. W. (1991) Induction of encephalitis in SJL mice by intranasal infection with herpes simplex virus type 1: a possible model of herpes simplex encephalitis in humans. J. Infect. Dis. 163, 720-727.

Kouri R. E., Salerno R A., and Whitmire C. E. (1973) Relationships between arylhydrocarbon hydroxylase inducibility and sensitivity to chemically induced subcutaneous sarcomas in various strains of mice. J. Natl. Cancer Inst. 50, 363-368.

Lammas D. A., Mitchell L. A., and Wakelin D. (1990) Genetic influences upon eosinophilia and resistance in mice infected with Mesocestoides corti. Parasitology 101, 291-299.

Levine B. B. and Vaz N. M. (1970) Effect of combinations of inbred strain, antigen and antigen dose on immune responsiveness and reagin production in the mouse. Int. Arch. Allergy 39, 156-171.

Levine S. and Sowinski R. (1973) Experimental allergic encephelomyelitis in inbred and outbred mice. J. Immunol. 110, 139-143.

Lieberman J. and Kellog F. (1967) Hyaline-membrane formation and pulmonary plasminogen-activator activity in various strains of mice. Pediatrics 39, 75-81.

Lindsey J. W. (1996) Characteristics of initial and reinduced experimental autoimmune encephalomyelitis. Immunogenet. 44, 292-297.

Lipton H. L. and Dal Canto M. C. (1976) Chronic neurologic disease in Theiler's virus infection of SJL/J mice. J. Neurol. Sci. 30, 201-207.

Lynch D. M. and Kay P. H. (1995) Studies on the polymorphism of the fifth component of complement in laboratory mice. Exp. Clin. Immunogenet 12, 253-260.

Maley M. A. L., Fan Y., Beilharz M. W., and Grounds M. D. (1994) Intrinsic differences in MyoD and myogenin expression between primary cultures of SJL/J and BALB/C skeletal muscle. Exp. Cell Research 211, 99-107.

McCarthy M. M. and Dutton R. W. (1975) The humoral response of mouse spleen cells to two types of sheep erythrocytes. J. Immunol. 115, 1316-1321.

Meier H. and MacPike A. D. (1968) Levels and heritability of serum ceruloplasmin activity in inbred strains of mice. Proc. Soc. Exp. Biol. Med. 128, 1185-1190.

Mitchell C. A., Grounds M. D., and Papadimitriou J. M. (1995) The genotype of bone marrow-derived inflammatory cells does not account for differences in skeletal muscle regeneration between SJL/J and BALB/c mice. Cell & Tissue Research 280, 407-413.

Murphy E. D. (1963) SJL/J, a new inbred strain of mouse with a high, early incidence of reticulum-cell neoplasms. Proc. Am. Assoc. Cancer Res. 4, 46.

Myers D. D., Meier H., and Huebner R. J. (1970) Prevalence of murine C-type RNA virus group specific antigen in inbred strains of mice. Life Sci. 9, 1071-1080.

Nishina P. M., Wang J., Toyofuku W., Kuypers F. A., Ishida B. Y., and Paigen B. (1993). Atherosclerosis and plasma and liver lipids in nine inbred strains of mice. Lipids 28, 599-605.

Noonan F. P. and Hoffman H. A. (1994) Susceptibility to immunosuppression by ultraviolet B radiation in the mouse. Immunogenet. 39, 29-39.

Owens H. M. and Bonavida B. (1976) Immune functions characteristic of SJL/J mice and their association with age and spontaneous reticulum cell sarcoma. Cancer Res. 36, 1077-1083.

Page D. L. and Glenner G. G. (1972) Social interaction and wounding in the genesis of spontaneous murine amyloidosis. Am. J. Pathol. 67, 555-570.

Perry L. L. and Lodmell D. L. (1991) Role of CD4+ and CD8+T cells in murine resistance to street rabies virus. J. Virol. 65, 3429-3434.

Pope B. L., Chourmouzis E., MacIntyre J. P., Lee S., and Goodman M. G. (1994) Murine strain variation in the natural killer cell and proliferative responses to the immunostimulatory compound 7-Allyl-8-oxoguanosine: Role of cytokines. Cell. Immunol. 159, 194-210.

Potter M. (1972) Immunoglobulin-producing tumors and myeloma proteins of mice. Physiol. Rev. 52,631-719.

Rager-Zisman B., Ju G., and Udem S. (1976) Resistance and susceptibility of mice to infection with measles virus. Fed. Proc. 35, 391.

Riven-Kreitman R., Tauber-Finkelstein M., Zipori D., and Shaltiel S. (1990) A periodicity in the response of SJL/J thymocytes to isoproterenol. Simulation by cell lines. Molecular & Cellular Endocrinology 73, 211-216.

Roderick T. H., Wimer R. E., Wimer C. C., and Schwartzkroin P. A. (1973) Genetic and phenotypic variation in weight of brain and spinal cord between inbred strains of mice. Brain Res. 64, 345-353.

Roderick T. H. (1963) The response of twenty-seven inbred strains of mice to daily doses of whole-body X-irradiation. Radiation Res. 20, 631-639.

Rubinstein P., Liu N., Strenn E. W., and Decary F. (1974) Electrophoretic mobility and agglutinability of red blood cells: a ‘new’ polymorphism in mice. J. Exp. Med. 139, 313-322.

Sakagami T., Dixon M., ORourke J., Howlett R., Alderuccio F., Vella J., Shimoyama T., and Lee A. (1996) Atrophic gastric changes in both Helicobacter felis and Helicobacter pylori infected mice are host dependent and separate from antral gastritis. Gut 39, 639-648.

Sampugna J., Clements J., Carter T. P., and Campagnoni A. T. (1975) Comparison of lipids in total brain tissue from five mouse genotypes. J. Neurobiol. 6, 259-266.

Schlager G. and Dickie M. M. (1967) Spontaneous mutations and mutation rates in the house mouse. Genetics 57, 319-330.

Shimosato K., Saito T., and Marley R. J. (1994) Genotype-specific blockade of cocaine-induced weight loss by the protein synthesis inhibitor, anisomycin. Life Sciences 55, PL293-PL299.

Smith A. M. (1976) The effects of age on the immune response to type III pneumococcal polysaccharide (SIII) and bacterial lipopolysaccharide (LPS) in BALB/c, SJL/J and C3H mice. J. Immunol. 116, 469-474.

Storer J. B. (1966) Longevity and gross pathology at death in 22 inbred strains of mice. J. Gerontol. 21, 404-409.

Storer J. B. (1967) Relation of lifespan to brain weight, body weight and metabolic rate among inbred mouse strains. Exp. Gerontol. 2, 173-182.

Takahashi M., Kleeberger S. R., and Croxton T. L. (1995) Genetic control of susceptibility to ozone-induced changes in mouse tracheal electrophysiology. American Journal of Physiology—Lung Cellular and Molecular Physiology 269, L6-L10.

Thomas P. E., Hutton J. J., and Taylor B. A. (1973) Genetic relationship between aryl hydrocarbon hydroxylase inducibility and chemical carcinogen induced skin ulceration in mice. Genetics 74, 655-659.

Treadwell P. E. (1969) The inheritance of susceptibility to anaphylaxis in inbred mice and their hybrid progenies. J. Reticuloendothel. Soc. 6, 343-353.

Wanebo H. J., Gallmier W. M., Boyse E. A., and Old L. J. (1966) Paraproteinemia and reticulum cell sarcoma in an inbred mouse strain. Science 154, 901-903.

Weibust R. S. (1973) Inheritance of plasma cholesterol levels in mice. Genetics 73, 303-312.

West D. B., Boozer C. N., Moody D. L., and Atkinson R. L. (1992) Dietary obesity in nine inbred mouse strains. Am. J. Physiol. 262, R1025-R1032.

Whitmire C. E., Salerno R. A., Rabstein L. S., Heubner R. J., and Turner H. C. (1971) RNA tumour-virus antigen expression in chemically induced tumours. Virus-genome specified common antigens detected by complement fixation in mouse tumours induced by 3-methylcholanthrene. J. Natl. Cancer Inst. 47, 1255-1265.

Williams R. W., Strom R. C., Rice D. S., and Goldowitz D. (1996) Genetic and environmental-control of variation in retinal ganglion-cell number in mice. Journal of Neuroscience 16, 7193-7205.

Yuhas J. M. and Storer J. B. (1969) On mouse strain differences in radiation resistance: hematopoietic death and the endogenous colony-forming unit. Radiation Res. 39, 608-622.

Zarrow M. X., Christenson C. M., and Eleftheriou B. C. (1971) Strain differences in the ovulatory response of immature mice to PMS and to the pheromonal facilitation of PMS-induced ovulation. Biol. Reprod. 4, 52-56.

Zhang L. Y., Levitt R. C., and Kleeberger S. R. (1995) Differential susceptibility to ozone-induced airways hyperreactivity in inbred strains of mice. Experimental

Prion References

Aguzzi, A.: Personal Communication. Zurich, Switzerland, Mar. 20, 1997.

Aguzzi, A.; Brandner, S. The genetics of prions-a contradiction in terms? Lancet 354: 22-25, 1999.

Aguzzi, A.; Weissmann, C. A suspicious signature. Nature 383: 666-667, 1996.

Basler, K.; Oesch, B.; Scott, M.; Westaway, D.; Walchli, M.; Groth, D. F.; McKinley, M. P.; Prusiner, S. B.; Weissmann, C. Scrapie and cellular PrP isoforms are encoded by the same chromosomal gene. Cell 46: 417-428, 1986.

Bertoni, J. M.; Brown, P.; Goldfarb, L. G.; Rubenstein, R.; Gajdusek, D. C. Familial Creutzfeldt-Jakob disease (codon 196 mutation) with supranuclear palsy. J.A.M.A. 268: 2413-2415, 1992.

Beyreuther, K.; Masters, C. L., Catching the culprit prion. Nature 370: 419-420, 1994.

Bosque, P. J.; Vnencak-Jones, C. L.; Johnson, M. D.; Whitlock, J. A.; McLean, M. J. A PrP gene codon 178 base substitution and a 24-bp interstitial deletion in familial Creutzfeldt-Jakob disease. Neurology 42: 1864-1870, 1992.

Brown, P.; Galvez, S.; Goldfarb, L. G.; Nieto, A.; Cartier, L.; Gibbs, C. J., Jr.; Gajdusek D. C. Familial Creutzfeldt-Jakob disease in Chile is associated with the codon 196 mutation of the PRNP amyloid precursor gene on chromosome 20. J. Neurol. Sci. 112: 65-67, 1992.

Brown, P.; Goldfarb, L. G.; Gajdusek, D. C. The new biology of spongiform encephalopathy: infectious amyloidoses with a genetic twist. Lancet 337: 1019-1022, 1991.

Brown, P.; Goldfarb, L. G.; Kovanen, J.; Haltia, M.; Cathala, F.; Sulima, M.; Gibbs, C. J., Jr.; Gajdusek, D. C. Phenotypic characteristics of familial Creutzfeldt-Jakob disease associated with the codon 178-asn PRNP mutation. Ann. Neurol. 31: 282-285, 1992.

Brown, P.; Goldfarb, L. G.; McCombie, W. R.; Nieto, A.; Squillacote, D.; Sheremata, W.; Little, B. W.; Godec, M. S.; Gibbs, C. J., Jr.; Gajdusek, D. C.: Atypical Creutzfeldt-Jakob disease in an American family with an insert mutation in the PRNP amyloid precursor gene. Neurology 42: 422-427, 1992.

Bueler, H.; Aguzzi, A.; Sailer, A.; Greiner, R.-A.; Autenried, P.; Aguet, M.; Weissman, C. Mice devoid of PrP are resistant to scrapie. Cell 73: 1339-1347, 1993.

Bueler, H.; Raeber, A.; Sailer, A.; Fischer, M.; Aguzzi, A.; Weissmann, C.: High prion and PrP(Sc) levels but delayed onset of disease in scrapie-inoculated mice heterozygous for a disrupted PrP gene. Molec. Med. 1: 19-30, 1994.

Campbell, T. A.; Palmer, M. S.; Will, R. G.; Gibb, W. R. G.; Luthert, P. J.; Collinge, J. A prion disease with a novel 96-base pair insertional mutation in the prion protein gene. Neurology 46: 761-766, 1996.

Carlson, G. A.; Kingsbury, D. T.; Goodman, P. A.; Coleman, S.; Marshall, S. T.; DeArmond, S.; Westaway, D.; Prusiner, S. B. Linkage of prion protein and scrapie incubation time genes. Cell 46: 503-511, 1986.

Chapman, J.; Arlazoroff, A.; Goldfarb, L. G.; Cervenakova, L.; Neufeld, M. Y.; Werber, E.; Herbert, M.; Brown, P.; Gajdusek, D. C.; Korczyn, A. D. Fatal insomnia in a case of familial Creutzfeldt-Jakob disease with the codon 196(lys) mutation. Neurology 46: 758-761, 1996.

Chapman, J.; Ben-Israel, J.; Goldhammer, Y.; Korczyn, A. D., The risk of developing Creutzfeldt-Jakob disease in subjects with the PRNP gene codon 196 point mutation. Neurology 44: 1683-1686, 1994.

Chapman, J.; Brown, P.; Rabey, J. M.; Goldfarb, L. G.; Inzelberg, R.; Gibbs, C. J., Jr.; Gajdusek, D. C.; Korczyn, A. D. Transmission of spongiform encephalopathy from a familial Creutzfeldt-Jakob disease patient of Jewish Libyan origin carrying the PRNP codon 196 mutation. Neurology 42: 1249-1250, 1992.

Chapman, J.; Korczyn, A. D. Genetic and environmental factors determining the development of Creutzfeldt-Jakob disease in Libyan Jews. Neuroepidemiology 10: 228-231, 1991.

Chiesa, R.; Drisaldi, B.; Quaglio, E.; Migheli, A.; Piccardo, P.; Ghetti, B.; Harris, D. A. Accumulation of protease-resistant prion protein (PrP) and apoptosis of cerebellar granule cells in transgenic mice expressing a PrP insertional mutation. Proc. Nat. Acad. Sci. 97: 5574-5579, 1960.

Chiesa, R.; Piccardo, P.; Ghetti, B.; Harris, D.: Neuron 21: 1339-1351, 1998.

Collinge, J. : Human prion diseases and bovine spongiform encephalopathy (BSE). Hum. Mol. Genet. 6: 1699-1705, 1997.

Collinge, J.; Brown, J.; Hardy, J.; Mullan, M.; Rossor, M. N.; Baker, H.; Crow, T. J.; Lofthouse, R.; Poulter, M.; Ridley, R.; Owen, F.; Bennett C.; Dunn, G.; Harding, A. E.; Quinn, N.; Doshi, B.; Roberts, G. W.; Honavar, M.; Janota, I.; Lantos, P. L.: Inherited prion disease with 144 base pair gene insertion. 2. Clinical and pathological features. Brain 115: 687-710, 1992.

Collinge, J.; Harding, A. E.; Owen, F.; Poulter, M.; Lofthouse, R.; Boughey, A. M.; Shah, T.; Crow, T. J.: Diagnosis of Gerstmann-Straussler syndrome in familial dementia with prion protein gene analysis. Lancet II: 15-17, 1989.

Collinge, J.; Owen, F.; Poulter, M.; Leach, M.; Crow, T. J.; Rossor, M. N.; Hardy, J.; Mullan, M. J.; Janota, I.; Lantos, P. L. : Prion dementia without characteristic pathology. Lancet 336: 7-9, 1990.

Collinge, J.; Palmer, M. S.; Dryden, A. J.: Genetic predisposition to iatrogenic Creutzfeldt-Jakob disease. Lancet337: 1441-1442, 1991.

Collinge, J.; Poulter, M.; Davis, M. B.; Baraitser, M.; Owen, F.; Crow, T. J.; Harding, A. E.: Presymptomatic detection or exclusion of prion protein gene defects in families with inherited prion diseases. Am. J. Hum. Genet. 49:1351-1354, 1991.

Collinge, J.; Sidle, K. C. L.; Heads, J.; Ironside, J.; Hill, A. F.: Molecular analysis of prion strain variation and the aetiology of ‘new variant’ CJD. Nature 383: 685-690, 1996.

Collinge, J.; Whittington, M.; Sidle, K. C. L.; Smith, C. J.; Palmer, M.; Clarke, A. R.; Jefferys, J. G. R.: Prion protein is necessary for normal synaptic function. (Letter) Nature 370: 295-297, 1994.

Colombo, R.: Age and origin of the PRNP E196K mutation causing familial Creutzfeldt-Jacob disease in Libyan Jews. (Letter) Am. J. Hum. Genet. 67: 528-531, 1960.

de Silva, R.; Ironside, J. W.; McCardle, L.; Esmonde, T.; Bell, J.; Will, R.; Windl, O.; Dempster, M.; Estibeiro, P.; Lathe, R.: Neuropathological phenotype and ‘prion protein’ genotype correlation in sporadic Creutzfeldt-Jakob disease. Neurosci. Lett. 179: 50-52, 1994.

Deslys, J.-P.; Jaegly, A.; d'Aignaux, J. H.; Mouthon, F.; de Villemeur, T. B.; Dormont, D.: Genotype at codon 129 and susceptibility to Creutzfeldt-Jakob disease. Lancet 351: 1251 only, 1998.

Dlouhy, S. R.; Hsiao, K.; Farlow, M. F.; Foroud, T.; Conneally, P. M.; Johnson, P.; Prusiner, S. B.; Hodes, M. E.; Ghetti, B.: Linkage of the Indiana kindred of Gerstmann-Straussler-Scheinker disease to the prion protein gene. Nature Genet. 1: 64-67, 1992.

Doh-ura, K.; Kitamoto, T.; Sakaki, Y.; Tateishi, J.: CJD discrepancy. (Letter) Nature 353: 801-802, 1991.

Doh-ura, K.; Tateishi, J.; Sasaki H.; Kitamoto, T.; Sakaki, Y.: Pro-to-leu change at position 102 of prion protein is the most common but not the sole mutation related to Gerstmann-Straussler syndrome. Biochem. Biophys. Res. Commun. 163: 974-979, 1989.

Duchen, L. W.; Poulter, M.; Harding, A. E.: Brain 116: 555-567, 1993.

Farlow, M. R.; Yee, R. D.; Dlouhy, S. R.; Conneally, P. M.; Azzarelli, B.; Ghetti, B. Gerstmann-Straussler-Scheinker disease. I. Extending the clinical spectrum. Neurology 39: 1446-1452, 1989.

Forloni, G.; Angeretti, N.; Chiesa, R.; Monzani, E.; Salmona, M.; Bugiani, O.: Neurotoxicity of a prion protein fragment. (Letter) Nature 362: 543-546, 1993.

Gabizon, R.; Rosenmann, H.; Meiner, Z.; Kahana, I.; Kahana, E.; Shugart, Y.; Ott, J.; Prusiner, S. B.: Mutation and polymorphism of the prion protein gene in Libyan Jews with Creutzfeldt-Jakob disease (CJD). Am. J. Hum. Genet. 53: 828-835, 1993.

Gajdusek, D. C.: The transmissible amyloidoses: genetical control of spontaneous generation of infectious amyloid proteins by nucleation of configurational change in host precursors: kuru-CJD-GSS-scrapie-BSE. Europ. J. Epidemiol. 7: 567-577, 1991.

Gambetti, P.; Petersen, R.; Monari, L.; Tabaton, M.; Autilio-Gambetti, L.: Fatal familial insomnia and the widening spectrum of prion diseases. Brit. Med. Bull. 49: 980-994, 1993.

Ghetti, B.; Tagliavini, F.; Masters, C. L.; Beyreuther, K.; Giaccone, G.; Verga, L.; Farlow, M. R; Conneally, P. M.; Dlouhy, S. R.; Azzarelli, B.; Bugiani, O.: Gerstmann-Straussler-Scheinker disease. II. Neurofibrillary tangles and plaques with PrP-amyloid coexist in an affected family. Neurology 39: 1453-1461, 1989.

Giaccone, G.; Verga, L.; Bugiani, O.; Frangione, B.; Serban, D.; Prusiner, S. B.; Farlow, M. R.; Ghetti, B.; Tagliavini, F.: Prion protein preamyloid and amyloid deposits in Gerstmann-Straussler-Scheinker disease, Indiana kindred. Proc. Nat. Acad. Sci. 89: 9349-9353, 1992.

Goldfarb, L. G.; Brown, P.; Cervenakova, L.; Gajdusek D. C.: Molecular genetic studies of Creutzfeldt-Jakob disease. Molec. Neurobiol. 8: 89-97,1994.

Goldfarb, L. G.; Brown, P.; Goldgaber, D.; Asher, D. M.; Rubenstein, R.; Brown, W. T.; Piccardo, P.; Kascsak, R. J.; Boellaard, J. W.; Gajdusek, D. C.: Creutzfeldt-Jakob disease and kuru patients lack a mutation consistently found in the Gerstmann-Straussler-Scheinker syndrome. Exp. Neurol. 108: 247-250, 1990.

Goldfarb, L. G.; Brown, P.; Haltia, M.; Cathala, F.; McCombie, W. R.; Kovanen, J.; Cervenakova, L.; Goldin, L.; Nieto, A.; Godec, M. S.; Asher, D. M.; Gajdusek, D. C.: Creutzfeldt-Jakob disease cosegregates with the codon 178-asn PRNP mutation in families of European origin. Ann. Neurol. 31: 274-281, 1992.

Goldfarb, L. G.; Brown, P.; McCombie, W. R.; Goldgaber, D.; Swergold, G. D.; Wills, P. R.; Cervenakova, L.; Baron, H.; Gibbs, C. J., Jr.; Gajdusek, D. C.: Transmissible familial Creutzfeldt-Jakob disease associated with five, seven, and eight extra octapeptide coding repeats in the PRNP gene. Proc. Nat. Acad. Sci. 88: 10926-10930, 1991.

Goldfarb, L. G.; Brown, P.; Mitrova, E., Cervenakova, L.; Goldin, L.; Korczyn, A. D.; Chapman, J.; Galvez, S.; Cartier, L.; Rubenstein, R.; Gajdusek, D. C.: Creutzfeldt-Jacob disease associated with the PRNP codon 196-lys mutation: an analysis of 45 families. Europ. J. Epidemiol. 7: 477-486, 1991.

Goldfarb, L. G.; Haltia, M.; Brown, P.; Nieto, A.; Kovanen, J.; McCombie, W. R; Trapp, S.; Gajdusek, D. C.: New mutation in scrapie amyloid precursor gene (at codon 178) in Finnish Creutzfeldt-Jakob kindred. (Letter) Lancet 337: 425, 1991.

Goldfarb, L. G.; Mitrova, E.; Brown, P.; Toh, B. H.; Gajdusek, D. C.: Mutation in codon 196 of scrapie amyloid protein gene in two clusters of Creutzfeldt-Jakob disease in Slovakia. (Letter) Lancet 336: 514-515, 1990.

Goldfarb, L. G.; Petersen, R. B.; Tabaton, M.; Brown, P.; LeBlanc, A. C.; Montagna, P.; Cortelli, P.; Julien, J.; Vital, C.; Pendelbury, W. W.; Haltia, M.; Wills, P. R.; Hauw, J. J.; McKeever, P. E.; Monari, L.; Schrank, B.; Swergold, G. D.; Autilio-Gambetti L.; Gajdusek, D. C.; Lugaresi, E.; Gambetti, P.: Fatal familial insomnia and familial Creutzfeldt-Jakob disease: disease phenotype determined by a DNA polymorphism. Science 258: 806-808, 1992.

Goldgaber, D.; Goldfarb, L. G.; Brown, P.; Asher, D. M.; Brown, W. T.; Lin, S.; Teener, J. W.; Feinstone, S. M.; Rubenstein, R.; Kascsak, R. J.; Boellaard, J. W.; Gajdusek, D. C.: Mutations in familial Creutzfeldt-Jakob disease and Gerstmann-Straussler-Scheinker's syndrome. Exp. Neurol. 106: 204-206, 1989.

Goldhammer, Y.; Gabizon, R.; Meiner, Z.; Sadeh, M.: An Israeli family with Gerstmann-Straussler-Scheinker disease manifesting the codon 102 mutation in the prion protein gene. Neurology 43: 2718-2719, 1993.

Griffith, J. S. : Self-replication and scrapie. Nature 215: 1043-1044, 1967.

Haltia, M.; Kovanen, J.; Goldfarb, L. G.; Brown, P.; Gajdusek, D. C.: Familial Creutzfeldt-Jakob disease in Finland: epidemiological, clinical, pathological and molecular genetic studies. Europ. J. Epidemiol. 7: 494-500, 1991.

Hegde, R. S.; Mastrianni, J. A.; Scott, M. R.; DeFea, K. A.; Tremblay, P.; Torchia, M.; DeArmond, S. J.; Prusiner, S. B.; Lingappa, V. R.: A transmembrane form of the prion protein in neurodegenerative disease. Science 279:827-834, 1998.

Hegde, R. S.; Tremblay, P.; Groth, D.; DeArmond, S. J.; Prusiner, S. B.; Lingappa, V. R.: Transmissible and genetic prion diseases share a common pathway of neurodegeneration. Nature 402: 732-736, 1999.

Horwich, A. L.; Weissman, J. S.: Deadly conformations—protein misfolding in prion disease. Cell 89: 499-510, 1997.

Hsiao, K.; Baker, H. F.; Crow, T. J.; Poulter, M.; Owen, F.; Terwilliger, J. D.; Westaway, D.; Ott; J.; Prusiner, S. B.: Linkage of a prion protein missense variant to Gerstmann-Straussler syndrome. Nature 338: 342-345, 1989.

Hsiao, K.; Cass, C.; Conneally, P. M.; Dlouhy, S. R.; Hodes, M. E.; Farlow, M. R.; Ghetti, B.; Prusiner, S. B.: Atypical Gerstmann-Straussler-Scheinker syndrome with neurofibrillary tangles: no mutation in the prion protein open-reading-frame in a patient of the Indiana kindred. (Abstract) Neurobiol. Aging 11: 302, 1990.

Hsiao, K.; Dlouhy, S. R.; Farlow, M. R.; Cass, C.; Da Costa, M.; Conneally, P. M.; Hodes, M. E.; Ghetti, B.; Prusiner, S. B.: Mutant prion proteins in Gerstmann-Straussler-Scheinker disease with neurofibrillary tangles. Nature Genet. 1: 68-71, 1992.

Hsiao, K.; Meiner, Z.; Kahana, E.; Cass, C.; Kahana, I.; Avrahami, D.; Scarlato, G.; Abramsky, O.; Prusiner, S. B.; Gabizon, R. : Mutation of the prion protein in Libyan Jews with Creutzfeldt-Jakob disease. New Eng. J. Med. 324: 1091-1097, 1991.

Hsiao, K. K.; Groth, D.; Scott, M.; Yang, S.-L.; Serban, H.; Rapp, D.; Foster, D.; Torchia, M.; DeArmond, S. J.; Prusiner, S. B.: Serial transmission in rodents of neurodegeneration from transgenic mice expressing mutant prion protein. Proc. Nat. Acad. Sci. 91: 9126-9130, 1994.

Ironside, J. W.; Sutherland, K.; Bell, J. E.; McCardle, L.; Barrie, C.; Estebeiro, K.; Zeidler, M.; Will, R. G.: A new variant of Creutzfeldt-Jakob disease: neuropathological and clinical features. Cold Spring Harbor Symp. Quant. Biol. 61: 523-530, 1996.

Jendroska, K.; Hoffmann, O.; Schelosky, L.; Lees, A. J.; Poewe, W.; Daniel, S. E.: Absence of disease related prion protein in neurodegenerative disorders presenting with Parkinson's syndrome. J. Neurol. Neurosurg. Psychiat. 57: 1249-1251, 1994.

Kitamoto, T.; Ohta, M.; Doh-ura, K.; Hitoshi, S.; Terao, Y.; Tateishi, J.: Novel missense variants of prion protein in Creutzfeldt-Jakob disease or Gerstmann-Straussler syndrome. Biochem. Biophys. Res. Commun. 191: 709-714, 1993.

Kocisko, D. A.; Come, J. H.; Priola, S. A.; Chesebro, B.; Raymond, G. J.; Lansbury, P. T.; Caughey, B.: Cell-free formation of protease-resistant prion protein. Nature 370: 471-474, 1994.

Krasemann, S.; Zerr, I.; Weber, T.; Poser, S.; Kretzschmar, H.; Hunsmann, G.; Bodemer, W.: Prion disease associated with a novel nine octapeptide repeat insertion in the PRNP gene. Molec. Brain Res. 34: 173-176, 1995.

Kretzschmar, H. A.; Neumann, M.; Stavrou, D.: Codon 178 mutation of the human prion protein gene in a German family (Backer family): sequencing data from 72-year-old celloidin-embedded brain tissue. Acta Neuropath. 89: 96-98, 1995.

Kretzschmar, H. A.; Stowring, L. E.; Westaway, D.; Stubblebine, W. H.; Prusiner, S. B.; DeArmond, S. J.: Molecular cloning of a human prion protein cDNA. DNA 5: 315-324, 1986.

Kuwahara, C.; Takeuchi, A. M.; Nishimura, T.; Haraguchi, K.; Kubosaki, A.; Matsumoto, Y.; Saeki, K.; Matsumoto, Y.; Yokoyama, T.; Itohara, S.; Onodera, T.: Prions prevent neuronal cell-line death. (Letter) Nature 400: 225-226, 1999.

Laplanche, J.-L.; El Hachimi, K. H.; Durieux, I.; Thuillet, P.; Defebvre, L.; Delasnerie-Laupretre, N.; Peoch, K.; Foncin, J.-F.; Destee, A.: Prominent psychiatric features and early onset in an inherited prion disease with a new insertional mutation in the prion protein gene. Brain 122: 2375-2386, 1999.

Laplanche, J. L.; Chatelain, J.; Thomas, S.; Launay, J. M.; Gaultier, C.; Derouesne, C.: Uncommon phenotype for a codon 178 mutation of the human PrP gene. (Letter) Ann. Neurol. 31: 345, 1992.

Lee, H. S.; Sambuughin, N.; Cervenakova, L.; Chapman, J.; Pocchiari, M.; Litvak, S.; Qi, H. Y.; Budka, H.; del Ser, T.; Furukawa, H.; Brown, P.; Gajdusek, D. C.; Long, J. C.; Korczyn, A. D.; Goldfarb, L. G.: Ancestral origins and worldwide distribution of the PRNP 196K mutation causing familial Creutzfeldt-Jakob disease. Am. J. Hum. Genet. 64: 1063-1070, 1999.

Liao, Y.-C. J.; Lebo, J.; Lebo, R V.; Clawson, G. A.; Smuckler, E. A.: Human prion protein cDNA: molecular cloning, chromosomal mapping, and biological implications. Science 233: 364-367, 1986.

Lindquist, S.: Mad cows meet Psi-chotic yeast: the expansion of the prion hypothesis. Cell 89: 495-498, 1997.

Little, B. W.; Brown, B. W.; Rodgers-Johnson, P.; Perl, D. P.; Gajdusek, D. C.: Familial myoclonic dementia masquerading as Creutzfeldt-Jakob disease. Ann. Neurol. 20: 231-239, 1986.

Lugaresi, E.; Medori, R; Montagna, P.; Baruzzi, A.; Cortelli, P.; Lugaresi, A.; Tinuper, P. Zucconi, M.; Gambetti, P.: Fatal familial insomnia and dysautonomia with selective degeneration of thalamic nuclei. New Eng. J. Med. 315: 997-1003, 1986.

Lugaresi, E.; Montagna, P.; Baruzzi, A.; Cortelli, P.; Tinuper, P.; Zucconi, M.; Gambetti, P. L.; Medori, R.: Insomnie familiale a evolution maligne: une nouvelle maladie thalamique. Rev. Neurol. 142: 791-792, 1986.

Mallucci, G. R.; Campbell, T. A.; Dickinson, A.; Beck, J.; Holt, M.; Plant, G.; de Pauw, K. W.; Hakin, R. N.; Clarke, C. E.; Howell, S.; Davies-Jones, G. A. B.; Lawden, M.; Smith, C. M. L.; Ince, P.; Ironside, J. W.; Bridges, L. R.; Dean, A.; Weeks, I.; Collinge, J.: Inherited prion disease with an alanine to valine mutation at codon 117 in the prion protein gene. Brain 122: 1823-1837, 1999.

Manetto, V.; Medori, R.; Cortelli, P.; Montagna, P.; Tinuper, P.; Baruzzi, A.; Rancurel, G.; Hauw, J.-J.; Vanderhaeghen, J.-J.; Mailleux, P.; Bugiani, O.; Tagliavini, F.; Bouras, C.; Rizzuto, N.; Lugaresi, E.; Gambetti, P.: Fatal familial insomnia: clinical and pathologic study of 5 new cases. Neurology 42: 312-319, 1992.

Manson, J. C.; Clarke, A. R.; McBride, P. A.; McConnell, I.; Hope, J.: PrP gene dosage determines the timing but not the final intensity or distribution of lesions in scrapie pathology. Neurodegeneration 3: 331-340, 1994.

Manuelidis, L.; Sklaviadis, T.; Manuelidis, E. E.: Evidence suggesting that PrP is not the infectious agent in Creutzfeldt-Jakob disease. EMBO J. 6: 341-347, 1987.

Mastrianni, J. A.; Curtis, M. T.; Oberholtzser, J. C.; Da Costa, M. M.; DeArmond, S.; Prusiner, S. B.; Garbern, J. Y.: Prion disease (PrP-A117V) presenting with ataxia instead of dementia. Neurology 45: 2042-2050, 1995.

Medori, R.: Personal Communication. New York, N. Y., May 17, 1990.

Medori, R.; Montagna, P.; Tritschler, H. J.; LeBlanc, A.; Cortelli, P.;. Tinuper, P.; Lugaresi, E.; Ganbetti, P.: Fatal familial insomnia: a second kindred with mutation of prion protein gene at codon 178. Neurology 42: 669-670, 1992.

Medori, R.; Tritschler, H.-J.: Prion protein gene analysis in three kindreds with fatal familial insomnia (FFI): codon 178 mutation and codon 129 polymorphism. Am. J. Hum. Genet. 53: 822-827, 1993.

Medori, R.; Tritschler, H.-J.; LeBlanc, A.; Villare, F.; Manetto, V.; Chen, H. Y.; Xue, R.; Leal, S.; Montagna, P.; Cortelli, P.; Tinuper, P.; Avoni, P.; Mochi, M.; Baruzzi, A.; Hauw, J. J.; Ott, J.; Lugaresi, E.; Autilio-Gambetti, L.; Gambetti, P.: Fatal familial insomnia, a prion disease with a mutation at codon 178 of the prion protein gene. New Eng. J. Med. 326: 444-449, 1992.

Meggendorfer, F.: Klinische und genealogische Beobachtungen bei einem Fall von spastischer Pseudosklerose Jakobs. Z. Ges. Neurol. Psychiat. 128: 337-341, 1930.

Meiner, Z.; Gabizon, R.; Prusiner, S. B.: Familial Creutzfeldt-Jakob disease: codon 196 prion disease in Libyan Jews. Medicine 76: 227-237, 1997.

Mestel, R.: Putting prions to the test. Science 273: 184-189, 1996.

Mitrova, E.; Lowenthal, A.; Appeal, B.: Familial Creutzfeldt-Jakob disease with temporal and spatial separation of affected members. Europ. J. Epidemiol. 6: 233-238, 1990.

Monari L.; Chen, S. G.; Brown, P.; Parchi, P.; Petersen, R. B.; Mikol, J.; Gray, F.; Cortelli, P.; Montagna, P.; Ghetti, B.; Goldfarb, L. G.; Gajdusek, D. C.; Lugaresi, E.; Gambetti, P.; Autilio-Gambetti, L.: Fatal familial insomnia and familial Creutzfeldt-Jakob disease: different prion proteins determined by a DNA polymorphism. Proc. Nat. Acad. Sci. 91: 2839-2842, 1994.

Montrasio, F.; Frigg, R.; Glatzel, M.; Klein, M. A.; Mackay, F.; Aguzzi, A.; Weissmann, C.: Impaired prion replication in spleens of mice lacking functional follicular dendritic cells. Science 288: 1257-1259, 1960.

Mouillet-Richard, S.; Ermonval, M.; Chebassier, C.; Laplanche, J. L.; Lehmann, S.; Launay, J. M.; Kellermann, O.: Signal transduction through prion protein. Science 289: 1925-1928, 1960.

Mouillet-Richard, S.; Teil, C.; Lenne, M.; Hugon, S.; Taleb, O.; Laplanche, J.-L.: Mutation at codon 210 (V210I) of the prion protein gene in a North African patient with Creutzfeldt-Jakob disease. J. Neurol. Sci. 168: 141-144, 1999.

Nieto, A.; Goldfarb, L. G.; Brown, P.; McCombie, W. R.; Trapp, S.; Asher, D. M.; Gajdusek, D. C.: Codon 178 mutation in ethnically diverse Creutzfeldt-Jakob disease families. (Letter) Lancet 337: 622-623, 1991.

Oesch, B.; Westaway, D.; Walchli, M.; McKinley, M. P.; Kent, S. B. H.; Aebersold, R.; Barry, R. A.; Tempst, P.; Teplow, D. B.; Hood,. L. E.; Prusiner, S. B.; Weissmann, C.: A cellular gene encodes scrapie PrP 27-30 protein. Cell 40: 735-746, 1985.

Owen, F.; Poulter, M.; Collinge, J.; Crow, T. J.: A codon 129 polymorphism in the PRIP gene. Nucleic Acids Res. 18: 3103, 1990.

Owen, F.; Poulter, M.; Collinge, J.; Crow, T. J.: Codon 129 changes in the prion protein gene in Caucasians. (Letter) Am. J. Hum. Genet. 46: 1215-1216, 1990.

Owen, F.; Poulter, M.; Collinge, J.; Leach, M.; Lofthouse, R.; Crow, T. J.; Harding, A. E.: Molec. Brain Res. 13: 155-157, 1992.

Owen, F.; Poulter, M.; Lofthouse, R.; Collinge, J.; Crow, T. J.; Risby, D.; Baker, H. F.; Ridley, R. M.; Hsiao, K.; Prusiner, S. B.: Insertion in prion protein gene in familial Creutzfeldt-Jakob disease. (Letter) Lancet I: 51-52, 1989.

Owen, F.; Poulter, M.; Shah, T.; Collinge, J.; Lofthouse, R.; Baker, H.; Ridley, R.; McVey, J.; Crow, T. J.: An in-frame insertion in the prion protein gene in familial Creutzfeldt-Jakob disease. Molec. Brain Res. 7: 273-276, 1990.

Pablos-Mendez, A.; Netto, E. M.; Defendini, R.: Infectious prions or cytotoxic metabolites? Lancet 341: 159-161, 1993.

Palmer, M. S.; Collinge, J.: Mutations and polymorphisms in the prion protein gene. Hum. Mutat. 2: 168-173, 1993.

Palmer, M. S.; Dryden, A. J.; Hughes, J. T.; Collinge, J.: Homozygous prion protein genotype predisposes to sporadic Creutzfeldt-Jakob disease. Nature 352: 340-342, 1991. Note: Erratum: Nature 352: 547 only, 1991.

Perry, R. T.; Go, R. C. P.; Harrell, L. E.; Acton, R. T.: SSCP analysis and sequencing of the human prion protein gene (PRNP) detects two different 24 bp deletions in an atypical Alzheimer's disease family. Am. J. Med. Genet. 60: 12-18, 1995.

Pocchiari, M.; Salvatore, M.; Cutruzzola, F.; Genuardi, M.; Allcatelli, C. T.; Masullo, C.; Macchi, G.; Alema, G.; Galgani, S.; Xi, Y. G.; Petraroli, R.; Silvestrini, M. C.; Brunori, M.: A new point mutation of the prion protein gene in Creutzfeldt-Jakob disease. Ann. Neurol. 34: 802-807, 1993.

Poulter, M.; Baker, H. F.; Frith, C. D.; Leach, M.; Lofthouse, R.; Ridley, R. M.; Shah, T.; Owen, F.; Collinge, J.; Brown, J.; Hardy, J.; Mullan, M. J.; Harding, A. E.; Bennett, C.; Doshi, R.; Crow, T. J.: Inherited prion disease with 144 base pair gene insertion. 1. Genealogical and molecular studies. Brain 115: 675-685, 1992.

Prusiner, S. B.: Prions causing degenerative neurological diseases. Annu. Rev. Med. 38: 381-398, 1987.

Prusiner, S. B.: Molecular biology of prion diseases. Science 252: 1515-1522, 1991.

Prusiner, S. B.: Biology and genetics of prion diseases. Ann. Rev. Microbiol. 48: 655-686, 1994.

Prusiner, S. B.: Novel proteinaceous infectious particles cause scrapie. Science 216: 136-144, 1982.

Prusiner, S. B.: Molecular biology and genetics of prion diseases. Cold Spring Harbor Symp. Quant. Biol. 61: 473-493, 1996.

Puckett, C.; Concannon, P.; Casey, C.; Hood, L.: Genomic structure of the human prion protein gene. Am. J. Hum. Genet. 49: 320-329,1991.

Reder, A. T.; Mednick, A. S.; Brown, P.; Spire, J. P.; Cauter, V.; Wollmann, R. L.; Cervenakova, L.; Goldfarb, L. G.; Garay, A.; Ovsiew, F.; Gajdusek, D. C.; Roos, R. P. Clinical and genetic studies of fatal familial insomnia. Neurology 45: 1068-1075, 1995.

Riek, R.; Wider, G.; Billeter, M.; Hornemann, S.; Glockshuber, R.; Wuthrich, K.: Prion protein NMR structure and familial human spongiform encephalopathies. Proc.Nat. Acad. Sci. 95: 11667-11672, 1998.

Rivera, H.; Zuffardi, O.; Maraschio, P.; Caiulo, A.; Anichini, C.; Scarinci, R.; Vivarelli, R.: Alternate centromere inactivation in a pseudodicentric (15;20)(pter;pter) associated with a progressive neurological disorder. J. Med. Genet. 26: 626-630, 1989.

Robakis, N. K.; Devine-Gage, E. A.; Jenkins, E. C.; Kascsak, R. J.; Brown, W. T.; Krawczun, M. S.; Silverman, W. P.: Localization of a human gene homologous to the PrP gene on the p arm of chromosome 20 and detection of PrP-related antigens in normal human brain. Biochem. Biophys. Res. Commun. 140: 758-765, 1986.

Sailer, A.; Bueler, H.; Fischer, M.; Aguzzi, A.; Weissmann, C.: No propagation of prions in mice devoid of PrP. Cell 77: 967-968, 1994.

Sakaguchi, S.; Katamine, S.; Nishida, N.; Moriuchi, R.; Shigamatsu, K.; Sugimoto, T.; Nakatani, A.; Kataoka, Y.; Houtani, T.; Shirabe, S.; Okada, H.; Hasegawa, S.; Miyamoto, T.; Noda, T.: Loss of cerebellar Purkinje cells in aged mice homoygous for a disrupted PrP gene. Nature 380: 528-531, 1996.

Samaia, H. B.; Mari, J. J.; Vallada, H. P.; Moura, R. P.; Simpson, A. J. G.; Brentani, R. R.: A prion-linked psychiatric disorder. Nature 390: 241 only, 1997.

Schellenberg, G. D.; Anderson, L.; O'dahl, S.; Wisjman, E. M.; Sadovnick, A. D.; Ball, M. J.; Larson, E. B.; Kukull, W. A.; Martin, G. M.; Roses, A. D.; Bird, T. D.: APP-717, APP-693, and PRIP gene mutations are rare in Alzheimer disease. Am. J. Hum. Genet. 49: 511-517, 1991.

Schnittger, S.; Gopal Rao, V. V. N.; Deutsch, U.; Gruss, P.; Balling, R.; Hansmann, I.: PAX1, a member of the paired box-containing class of developmental control genes, is mapped to human chromosome 20p11.2 by in situ hybridization (ISH and FISH). Genomics 14: 740-744, 1992.

Scott M.; Foster, D.; Mirenda, C.; Serban, D.; Coufal, F.; Walchli, M.; Torchia, M.; Groth, D.; Carlson, G.; DeArmond, S. J.; Westaway, D.; Prusiner, S. B.: Transgenic mice expressing hamster prion protein produce species-specific scrapie infectivity and amyloid plaques. Cell 59: 847-857, 1989.

Shibuya, S.; Higuchi, J.; Shin, R.-W.; Tateishi, J.; Kitamoto, T.: Protective prion protein polymorphisms against sporadic Creutzfeldt-Jakob disease. (Letter) Lancet 351: 419 only, 1998.

Shmerling, D.; Hegyi, I.; Fischer, M.; Blattler, T.; Brandner, S.; Gotz, J.; Rulicke, T.; Flechsig, E.; Cozzio, A.; von Mering, C.; Hangartner, C.; Aguzzi, A.; Weissmann, C.: Expression of amino-terminally truncated PrP in the mouse leading to ataxia and specific cerebellar lesions. Cell 93: 203-214, 1998.

Simon, E. S.; Kahana, E.; Chapman, J.; Treves, T. A.; Gabizon, R.; Rosenmann, H.; Zilber, N.; Korczyn, A. D.: Creutzfeldt-Jakob disease profile in patients homozygous for the PRNP E196K mutation. Ann. Neurol. 47: 257-260, 1960.

Sparkes, R. S.; Simon, M.; Cohn, V. H.; Fournier, R. E. K.; Lem, J.; Klisak, I.; Heinzmann, C.; Blatt, C.; Lucero, M.; Mohandas, T.; DeArmond, S. J.; Westaway, D.; Prusiner, S. B.; Weiner, L. P.: Assignment of the human and mouse prion protein genes to homologous chromosomes. Proc. Nat. Acad. Sci. 83: 7358-7362, 1986.

Speer, M. C.; Goldgaber, D.; Goldfarb, L. G.; Roses, A. D.; Pericak-Vance, M. A.: Support of linkage of Gerstmann-Straussler-Scheinker syndrome to the prion protein gene on chromosome 20p12-pter. Genomics 9: 366-368, 1991.

Supattapone, S.; Bosque, P.; Muramoto, T.; Wille, H.; Aagaard, C.; Peretz, D.; Nguyen, H.-O. B.; Heinrich, C.; Torchia, M.; Safar, J.; Cohen, F. E.; DeArmond, S. J.; Prusiner, S. B.; Scott, M.: Prion protein of 106 residues creates an artificial transmission barrier for prion replication in transgenic mice. Cell 96: 869-878, 1999.

Tagliavini, F.; Prelli, F.; Ghiso, J.; Bugiani, O.; Serban, D.; Prusiner, S. B.; Farlow, M. R.; Ghetti, B.; Frangione, B.: Amyloid protein of Gerstmann-Straussler-Scheinker disease (Indiana kindred) is an 11 kd fragment of prion protein with an N-terminal glycine at codon 58. EMBO J. 10: 513-519, 1991.

Tagliavini, F.; Prelli, F.; Porro, M.; Rossi, G.; Giaccone, G.; Farlow, M. R.; Diouhy, S. R.; Ghetti, B.; Bugiani, O.; Frangione, B.: Amyloid fibrils in Gerstmann-Straussler-Scheinker disease (Indiana and Swedish kindreds) express only PrP peptides encoded by the mutant allele. Cell 79: 695-703, 1994.

Tateishi, J.; Brown, P.; Kitamoto, T.; Hoque, Z. M.; Roos, R.; Wollman, R.; Cervenakova, L.; Gajdusek, D. C.: First experimental transnission of fatal familial insomnia. Nature 376: 434-435, 1995.

Telling, G. C.; Parchi, P.; DeArmond, S. J.; Cortelli, P.; Montagna, P.; Gabizon, R.; Mastrianni, J.; Lugaresi, E.; Gambetti, P.; Prusiner, S. B.: Evidence for the conformation of the pathologic isoform of the prion protein enciphering and propagating prion diversity. Science 274: 2079-1962, 1996.

Telling, G. C.; Scott, M.; Mastrianni, J.; Gabizon, R.; Torchia, M.; Cohen, F. E.; DeArmond, S. J.; Prusiner, S. B.: Prion propagation in mice expressing human and chimeric PrP transgenes implicates the interaction of cellular PrP with another protein. Cell 83: 79-90, 1995.

Ter-Avanesyan, M. D.; Dagkesamanskaya, A. R.; Kushnirov, V. V.; Srirnov, V. N.: The SUP35 omnipotent suppressor gene is involved in the maintenance of the non-mendelian determinant [psi+] in the yeast Saccharomyces cerevisiae. Genetics 137: 671-676, 1994.

Tobler, I.; Gaus, S. E.; Deboer, T.; Ackermann, P.; Fischer, M.; Rullcke, T.; Moser, M.; Oesch, B.; McBride, P. A.; Manson, J. C.: Altered circadian activity rhythms and sleep in mice devoid of prion protein. Nature 380: 639-642, 1996.

Westaway, D.; DeArmond, S. J.; Cayetano-Canlas, J.; Groth, D.; Foster, D.; Yang, S.-L.; Torchia, M.; Carlson, G. A.; Prusiner, S. B.: Degeneration of skeletal muscle, peripheral nerves, and the central nervous system in transgenic mice overexpressing wild-type prion proteins. Cell 76: 117-129, 1994.

Whittington, M. A.; Sidle, K. C. L.; Gowland, I.; Meads, J.; Hill, A. F.; Palmer, M. S, Jefferys, J. G. R.; Collinge, J.: Rescue of neurophysiological phenotype seen in PrP null mice by transgene encoding human prion protein. Nature Genet. 9: 197-201, 1995.

Wickner, R. B.: [URE3] as an altered URE2 protein: evidence for a prior analog in Saccharomyces cerevisiae. Science 264: 566-569, 1994.

Windl, O.; Giese, A.; Schulz-Schaeffer, W.; Zerr, I.; Skworc, K.; Arendt, S.; Oberdieck, C.; Bodemer, M.; Poser, S.; Kretzschmar, H. A.: Molecular genetics of human prion diseases in Germany. Hum. Genet. 105: 244-252, 1999.

Yamada, M.; Itoh, Y.; Fujigasaki, H.; Naruse, S.; Kaneko, K.; Kitamoto, T.; Tateishi, J.; Otomo, E.; Hayakawa, M.; Tanaka, J.; Matsushita, M.; Miyatake, T.: A missense mutation at codon 105 with codon 129 polymorphism of the prion protein gene in a new variant of Gerstmann-Straussler-Scheinker disease. Neurology 43: 2723-2724, 1993.

Species Barrier References

Collinge J, Palmer M S, Sidle K C, Hill A F, Gowland I, Meads J, Asante E, Bradley R, Doey L J, Lantos P L. (1995). Nature 378:779-83.

Collinge J, Sidle K C, Meads J, Ironside J, Hill A F. (1996) Nature. 383:685-90.

Hecker R, Taraboulos A, Scott M, Pan K M, Yang S L, Torchia M, Jendroska K, DeArmond S J, Prusiner S B. (1992) Genes Dev. 7:1213-28.

Hill A F, Desbruslais M, Joiner S, Sidle K C, Gowland I, Collinge J, Doey L J, Lantos P. (1997)Nature. 389:448-50.

Hill A F, Joiner S, Linehan J, Desbruslais M, Lantos P L, Collinge J. (1960). Proc Natl Acad Sci USA. 18:10248-53

Telling G C, Scott M, Mastrianni J, Gabizon R, Torchia M, Cohen F E, DeArmond S J, Prusiner S B. (1995). Cell 83(1):79-90.

1. A method for the detection of prions in a sample comprising the steps of:
(a) contacting one or more test animals with the sample;
(b) incubating the test animals;
(c) monitoring the test animals for adverse effects or death; and optionally
(d) performing a biopsy on the test animals that display adverse effects or death for evidence of prions; wherein the test animals have prion incubation times of 196 days or less.

2. A method according to claim 1 wherein the test animals to be contacted with the sample are mice.

3. A method according to claim 2 wherein the test animals to be contacted with the sample are derived from SJL mice.

4. A method according to claim 3 wherein the test animals to be contacted with the sample are SJL mice.

5. A method according to claim 1 wherein the test animals to be contacted with the sample are transgenic for one or more gene(s) from SJL mice.

6. A method according to claim 5 wherein the gene(s) comprise one or more PrP gene(s).

7. A method according to claim 6 wherein the PrP gene(s) encode a mammalian PrP.

8. A method according to claim 7 wherein the PrP gene(s) encode a livestock or a human PrP.

9. A method according to claim 1 wherein the sample contains prions that would cause BSE or vCJD in their appropriate host.

10. A method according to claim 9 wherein the sample is derived from a mammal.

11. A method according to claim 10 wherein the sample is derived from livestock or a human.

12. A method for the identification of genes associated with short prion incubation times comprising the steps of:
(a) contacting one or more test animals with a sample;
(b) incubating the test animals;
(c) monitoring the test animals for adverse effects or death; and optionally
(d) performing a biopsy on the test animals that display adverse effects or death for evidence of prions;
(e) identifying test animals with short and with longer prion incubation times;
(f) comparing the genes of test animals with short and with longer prion incubation times;
(g) identifying one or more genes that differ in the test animals with short and with longer prion incubation times; and optionally
(h) determining the function of one or more genes that differ between the test animals with short and with longer prion incubation times.

13. A method according to claim 12 wherein the test animals to be contacted with the sample are transgenic for one or more prion susceptibility genes.

14. A method for identifying one or more agents capable of modulating prion infection comprising the steps of:
(a) contacting one or more test animals with a sample;
(b) contacting one or more test animals with an agent;
(c) incubating the test animals;
(d) monitoring the test animals for adverse effects or death and performing a biopsy on the test animals that display adverse effects or death for evidence of prions;
(e) identifying said agents that increase or decrease the prion incubation time, wherein optionally a biopsy on the test animals that display adverse effects or death for evidence of prions may be performed after step (d).

15. An agent identified by a method according to claim 14 wherein said agent is capable of modulating prion infection.

16. A medicament for the treatment of a prion infection comprising an agent according to claim 15.

17. A method of modulating prion infection in a subject comprising administering to said subject a therapeutically effective amount of an agent according to claim 15.

18. A method for estimating the amount of prions in a sample comprising the steps of:
(a) contacting one or more test animals with the sample;
(b) incubating the test animals;
(c) monitoring the test animals;
(d) noting the amount of time taken for the test animal to display clinical symptoms of prion infection and the amount of time taken for the test animal to die;
(f) estimating the amount of prions in the sample from the times of(d).

19. A method of estimating the susceptibility of test animals to prion infection, comprising the steps of:
(a) incubating the test animals;
(b) monitoring the test animals for adverse effects or death;
(d) performing a biopsy on the test animals that display adverse effects or death for evidence of prions;
(e) performing glycoform ratio analysis;
(f) estimating the susceptibility of test animals to prion infection.

20. A method according to claim 21 wherein the sample contains prions that cause BSE or vCJD in their appropriate host.

21. A method according to claim 1 wherein the test animals have prion incubation times of 100 days or less.

22. A method according to claim 1 wherein the test animals have prion incubation times of 40 days or less.

23. A pharmaceutical composition comprising an agent according to claim 15.

24. A method according to claim 12 wherein the test animals have prion incubation times of 100 days or less.

25. A method according to claim 12 wherein the test animals have prion incubation times of 40 days or less.

26. A method according to claim 14 wherein the test animals have prion incubation times of 100 days or less.

27. A method according to claim 14 wherein the test animals have prion incubation times of 40 days or less.

28. A method according to claim 18 wherein the test animals have prion incubation times of 100 days or less.

29. A method according to claim 18 wherein the test animals have prion incubation times of 40 days or less.

30. A method according to claim 19 wherein the test animals have prion incubation times of 100 days or less.

31. A method according to claim 19 wherein the test animals have prion incubation times of 40 days or less.
